(19) United States
(12) Patent Application Publication
(10) Pub. No.: US 2022/0241400 A1
(43) Pub. Date: Aug. 4, 2022

(54) IMMUNOMODULATION PLATFORM AND METHODS OF USE

(71) Applicant: PROVAXUS, INC., Dover, DE (US)

(72) Inventors: Viktor Stolc, Dover, DE (US); Kevin White, Dover, DE (US)

(21) Appl. No.: 17/589,427

(22) Filed: Jan. 31, 2022

Related U.S. Application Data
(60) Provisional application No. 63/144,601, filed on Feb. 2, 2021.

(51) Int. Cl. A61K 39/215; C07K 14/005; A61P 37/04; A23L 33/135; A23C 9/123; A23C 19/00; A23G 9/36

(52) U.S. Cl. CPC A61K 39/215; C07K 14/005; A61P 37/04; A23L 33/135; A23C 9/1234; A61K 2039/523; A23G 9/363; C12N 2770/20022; C12N 2770/20023; C12N 2770/20034; C12N 2770/20071; A23C 19/00

(57) ABSTRACT

Disclosed herein are methods, systems, and compositions comprising genetically modified probiotic microorganisms. In some embodiments, the genetically modified probiotic microorganisms produce at least one viral coat protein and/or at least one fusion protein comprising an antigenic polypeptide linked to a viral coat protein.

### CROSS-REFERENCE TO RELATED APPLICATIONS

This application claims the benefit of priority under 35 U.S.C. § 119(e) of U.S. Provisional Application No. 63/144,601, filed Feb. 2, 2021, the contents of which is incorporated herein by reference in its entirety.

### SEQUENCE LISTING

A Sequence Listing accompanies this application and is submitted as an ASCII text file of the sequence listing named “174700_00015_ST25.txt” which is 18,381 bytes in size and was created on Jan. 31, 2022. The sequence listing is electronically submitted via EFS-Web with the application and is incorporated herein by reference in its entirety.

### FIELD

The present disclosure relates to the fields of molecular biology, virology, immunology and medicine. The disclosure provides a recombinant bacterium, the recombinant bacterium being genetically modified to produce at least one antigenic polypeptide comprising, for example, a virus-like particle (VLP), or a fusion protein comprising a VLP linked to at least one additional antigenic polypeptide. In some embodiments, the VLP or the VLP-fusion protein is recombinantly produced in a host probiotic edible bacterium, such as Lactobacillus acidophilus, to produce an antigen capable of modulating the immune system, including but not limited to acting as a vaccine. Also provided herein are compositions comprising a virus-like particle (VLP) of an RNA-bacteriophage, and/or comprising a fusion protein, comprising a VLP linked to at least one antigen.

### BACKGROUND

The GI tract is a complex and dynamic ecosystem containing a diverse collection of microorganisms [1]. The vast majority of microbial cells in the human GI tract are bacteria by belonging to, at the phylum-level, two phyla, the Bacteroidetes and the Firmicutes, although other phyla are present. Host physiology and intestinal microbiota are intimately connected. This is evident from the fact that each distinct anatomical region along the GI tract is characterized by its own physiochemical conditions, and that these changing conditions exert a selective pressure on the microbiota. The physiochemical conditions that influence the composition of the intestinal microbiota include: intestinal motility, pH, redox potential, nutrient supplies, host secretions (e.g. hydrochloric acid, digestive enzymes, bile and mucus), and the presence of an intact ileocecal valve. Thus, the GI tract harbors many distinct niches, each containing a different microbial ecosystem that varies according to the location within the GI tract. This is already demonstrated by the fact that the microbial density increases along the GI tract. Indeed, per gram of intestinal content, the microbial density increases from 10¹-10⁴ microbial cells in the stomach and duodenum, 10⁴-10⁸ cells in the jejunum and ileum, to 10¹⁰ - 40¹² cells in the colon and feces.

Recently, the collective genome of the human intestinal microbiome (IM) was estimated to contain 3.3 million microbial genes, which is about 150 times more genes than the human genome [2]. The presence of this wide array of genes in addition to our own genome, suggests that a profound influence of intestinal microorganisms on the human body can be expected.

The IM plays an important role in metabolic, nutritional, physiological and immunological processes in the human body [3]. It exerts important metabolic activities by extracting energy from otherwise indigestible dietary polysaccharides such as resistant starch and dietary fibers. These metabolic activities also lead to the production of fundamental nutrients such as short-chain fatty acids (SCFA), vitamins (e.g. vitamin K, vitamin B12 and folic acid) and amino acids, which humans are unable to produce by themselves.

Another important role of the IM is that it is involved in the defense against pathogens through mechanisms such as colonization resistance and production of antimicrobial compounds [43]. In addition, the IM participates in the development, maturation and maintenance of the GI sensory and motoric functions, the intestinal barrier and the mucosal immune system.

Since it is known that the IM plays an important role in human health and disease, manipulation of these microorganisms by probiotics, prebiotics and synbiotics are attractive approaches to improve and maintain health. According to the definition formulated by the World Health Organization (WHO) probiotics are “living microorganisms which, when administered in adequate amounts, confer a health benefit on the host [4]. Moreover, prebiotics are used to manipulate the microbiota composition in the GI tract. The definition of prebiotics is even more generic than the one of probiotics: “non-digestible food ingredients that, when consumed in sufficient amounts, selectively stimulate the growth and/or activity(ies) of one or a limited number of microbial genus(era)/species in the gut microbiota that confer(s) health benefits to the host”. Mixture of both probiotics and prebiotics are referred to as synbiotics.

Numerous health-beneficial effects have been attributed to probiotic microorganisms. In general, these health benefits can be categorized into three levels of probiotic action [5]. First, probiotic microorganisms can act directly with the GI tract (level 1), for example by direct interaction with the IM or by enzymatic activities. Second, they can interact directly with the intestinal mucus layer and epithelium (level 2), thereby influencing the intestinal barrier function and the mucosal immune system. Third, probiotics can have effects outside the GI tract (level 3), for example on the systemic immune system and other organs, such as liver and brain.

A role for the IM in the pathogenesis of several diseases and disorders has been suggested [6]. Multiple studies in the recent years hypothesize that the microbiome is critically important for normal host functions, while impaired host microbiome interactions contribute to the pathogenesis of numerous common disorders. Of these, much attention is recently given to the involvement of microbiome in the pathogenesis of impaired glucose tolerance, type 2 diabetes mellitus (T2DM), and other metabolic disorders comprising metabolic syndrome (MetS), including obesity, non-alcoholic fatty liver disease, and related complications [7].

Since modulation of the composition of intestinal microbiota by probiotics/prebiotics was demonstrated to be possible, probiotic/prebiotic consumption has become the norm in society. Probiotics are consumed in the form of dietary supplements and in foods such as yogurt, kefir, tempeh, sauerkraut, and kimchi, which are touted for their probiotic health benefits.

In addition to their use in supporting general health and immunity, probiotics have also been investigated as vaccine adjuvants and vaccine delivery system [8]. The development of improved vaccination strategies has always been of the utmost importance for a number of reasons.

First is the need to provide better levels of immunity against pathogens which enter the body primarily through the mucosal surfaces. Vaccines are generally given parenterally. However, many diseases use the gastrointestinal (GI) tract as the primary portal of entry. Thus, cholera and typhoid are caused by ingestion of the pathogens Salmonella typhi and Vibrio cholera and subsequent colonization at (V. cholera) or translocation (S. typhi) across the mucosal epithelium (lining the GI tract) [9]. Similarly, TB is initially caused by infection of the lungs by Mycobacterium tuberculosis [10]. Immunization via an injection generates a serum response (humoral immunity) which includes a predominant IgG response which is least effective in preventing infection. This is one reason why many vaccines are partially effective or give short protection times [11].

Second, is the need to provide a needle-less routes of administration. A major problem of current vaccination programs is that they require at least one injection. One example is tetanus vaccine. Although protection lasts for 10 years, children are initially given three doses by injection followed by a booster every 5 years [44]. Many people will choose not to take boosters because of fear of injection. In contrast, in developing countries where mortality from tetanus is high the problems often lie with using needles that are re-used or are not sterile.

Third, is the need to offer improved safety and minimize adverse side effects. Many vaccines consist of either live organisms which are either rendered non-pathogenic (attenuated) or are inactivated in some way. While in principle, this is considered safe there is evidence showing that safer methods must be developed. For example, in 1949 (the Kyoto incident) 68 children died from receiving a contaminated diphtheria vaccine. Likewise, in the Cutter incident of 1995 105 children developed polio. It was found that the polio vaccine had not been correctly inactivated with formalin. Many other vaccines, for example the MMR (measles-mumps-rubella) vaccine and the whooping cough vaccine are plagued with rumors of side effects, such as autism spectrum and autoimmunity [12].

Fourth is the need to provide economic vaccines for developing countries where poor storage and transportation facilities prevent effective immunization programs. In developing countries where a vaccine must be imported it is assumed that the vaccine will be stored and distributed correctly. The associated costs of maintaining vaccines in proper hygienic conditions under refrigeration are significant for a developing country. For some vaccines such as the oral polio vaccine and BCG vaccine the vaccines will only survive for one year at 2-8° C. [13]. The need for a robust vaccine that can be stored for extended periods at ambient temperature is a high priority now for developing countries. This type of vaccine should ideally be stable at ambient temperature, able to withstand variations in temperature as well as desiccation. Finally, a vaccine that is simple to produce would offer enormous advantages to a developing country and would potentially be producible in that country.

Accordingly, there is a need in the art to develop robust, vaccine platforms for oral delivery [14].

### SUMMARY

Disclosed herein are methods, systems, and compositions comprising genetically modified probiotic microorganisms. In some embodiments, the genetically modified probiotic microorganisms produce at least one viral coat protein and/or at least one fusion protein comprising an antigenic polypeptide linked to a viral coat protein.

Also disclosed herein are methods and compositions comprising a novel vaccine delivery system. In some embodiments, the vaccine delivery system includes a genetically modified probiotic microorganism engineered to produce at least one fusion protein comprising an antigenic polypeptide linked to a viral coat protein.

Also disclosed herein are methods and compositions comprising a genetically modified probiotic microorganism engineered to produce a viral coat protein alone. In some embodiments, the genetically modified probiotic microorganism is formulated as a vaccine.

In some embodiments, a modified probiotic microorganism is provided herein. In some embodiments, the modified microorganism comprises a nucleic acid sequence encoding a heterologous protein comprising one or more of: a viral coat protein; and a fusion of an antigenic peptide and a viral coat protein. In some embodiments, the modified probiotic microorganism comprises bacteria selected from Lactobacillus, Saccharomyces, Bifidobacterium, Streptococcus, Escherichia coli, and Bacillus, Leuconostoc, Pediococcus, Lactococcus, Aerococcus, Carnobacterium, Enterococcus, Oenococcus, Sporolactobacillus, Teragenococcus, Vagococcus, Weisella and other such bacteria related by genome sequence. In some embodiments, the modified probiotic microorganism comprises a Lactobacillus selected Lactobacillus acidophilus, Lactobacillus crispatus, Lactobacillus gasseri, Lactobacillus delbreuckii, Lactobacillus rhamnosus, Lactobacillus salivarius, Lactobacillus paracasei, Lactobacillus reuteri, Lactobacillus bulgaricus, Lactobacillus casei, Lactobacillus lactis, Lactobacillus plantarum, Lactobacillus rhamnosus, Bifidobacterium longum, Bifidobacterium breve, Bifidobacterium lactis, Lactobacillus reuterior and Lactobacillus fermentum and other such bacteria related by genome sequence. In some embodiments, the modified probiotic microorganism comprises Lactobacillus acidophilus.

In some embodiments, the nucleic acid sequence encoding the heterologous protein is integrated into the genome of the microorganism or is encoded on a plasmid or a vector within the microorganism. For example, in some embodiments, the nucleic acid sequence encoding the heterologous protein is integrated into the uracil phosphoribosyltransferase (upp) gene of the microorganism or at other suitable genome loci. In some embodiments, the modified probiotic microorganism expresses the heterologous protein, and the expressed protein self-assembles to form virus-like particles (VLPs). In some embodiments, the modified microorganism comprising VLPs. In some embodiments, the heterologous nucleic acid encodes a fusion of an antigenic peptide and a viral coat protein, and the fusion protein self-assembles to form VLPs. In some embodiments, the heterologous nucleic acid encodes a fusion of an antigenic peptide and a viral coat protein, and the expressed protein does not self-assemble to form VLPs.

In some embodiments, the nucleic acid sequence encoding a fusion protein further comprises one or more of the following: a linker sequence joining the antigenic peptide and coat protein; and an immunostimulatory sequence.

In some embodiments, the viral coat protein comprises one or more of the PP7, MS2, AP205, Qβ, R17, SP, PP7, GA, M11, MX1, f4, CbS, Cb12r, Cb23r, 7s and f2 coat proteins, or other VLP-forming proteins. For example, in some embodiments, the viral coat protein comprises the bacteriophage AP205 coat protein.

In some embodiments, the modified probiotic microorganism is live in culture, in spore form, or inactivated. In some embodiments, the microorganism is dead or is lyophilized.

Also disclosed herein are nutritional or therapeutic composition comprising the probiotic microorganism as described above. In some embodiments, the composition is formulated as a food or beverage or otherwise incorporated into the food supply. In some embodiments, the food or beverage comprises a dairy product, for example, milk, yogurt, cheese, or ice cream. In some embodiments, the composition is formulated as a capsule, powder, table, liquid, or sachet for oral administration. In some embodiments, the composition is formulated for nasal, rectal, parenteral, or transmucosal delivery.

Disclosed herein are methods for vaccinating a subject comprising: administering an effective amount of a therapeutic composition as described above. Also disclosed herein are methods of providing a nutritional supplementation to subject, comprising administering a nutritionally effective amount of a composition as described above. In some embodiments, administration comprises oral administration. In some embodiments, administration comprises nasal, rectal, parenteral, or transmucosal delivery. In some embodiments, a therapeutically effective amount or a nutritionally effective amount is provided as one or more doses. In some embodiments, a therapeutically or nutritionally effective amount is provided by administering multiple doses over the course of a week, two weeks, three weeks, or a month. In some embodiments a therapeutically or nutritionally effective amount is provided as a single dose in a single administration.

These and other embodiments, aspects, advantages, and features of the present invention will be set forth in part in the description which follows and will become apparent to those skilled in the art by reference to the following description of the invention and referenced drawings or by practice of the invention. The accompanying drawings illustrate one or more implementations, and these implementations do not necessarily represent the full scope of the invention.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, where:

FIG. 1. Shows the structure or an exemplary AP205 virus-like particle surface, containing a self-assembled complex of the coat protein and exposing vaccine antigenic peptides on its surface. In this example, antigenic peptides comprising at least one epitope are linked to both the C- and the N-terminus (shown in red and blue) of each copy of the coat protein.

FIG. 2. Provides a diagram of a plasmid-based homologous recombination construct. In this example, the uracil phosphoribosyltransferase gene (upp gene) of the probiotic microorganism (top) is targeted for insertion of the antigen-VLP (Provaxus VLP-encoding platform) sequence (bottom). The exemplary plasmid includes homologous sequences (“up” and “down”) of the target insertion site. The “up” and “down” homologous sequences flank the antigen-VLP sequences to be inserted. The plasmid in this example also includes an antibiotic resistance gene (ABR) for growth selection and the recT gene to facilitate recombination between the plasmid sequence and the host gene.

FIG. 3A-B. Provide exemplary plasmids without (A) and with (B) the recT gene.

FIG. 4A-B. Provide BLAST analysis of the recombination sequences in the upp gene, demonstrating the feasibility of a gene replacement strategy across many strains. (A) 100% identity with Lactobacillus acidophilus NCFM (SEQ ID NO: 26). (B) 86-89% identity with Lactobacillus crispatus strain STI. The base sequence is from L. acidophilius (SEQ ID NO: 27). While the figure demonstrates that the upp sequence and homologues in various species are suitable for gene replacement/homologous recombination strategies, other genetic loci are equally suitable as targets, and it is understood that the present methods and compositions are not intended to be limited by gene replacement targets, sequences used therein, or specific gene replacement methods.

FIG. 5A-C. Shows the structure of three exemplary antigen-CP fusion proteins. (A) shows a CP-fusion protein monomer having a single antigen linked to one end (either the C-terminus or the N-terminus) of the CP. (B) shows an antigen-CP fusion protein monomer having two identical antigen sequences linked to a different end of the CP (each of the C-terminus and N-terminus of the CP). (C) shows an antigen-CP fusion protein monomer having two different antigen sequences, each antigen sequence linked to a different end (each of the C-terminus and N-terminus) of the CP.

### DETAILED DESCRIPTION

The present invention is described herein using several definitions, as set forth below and throughout the application.

Unless otherwise specified or indicated by context, the terms “a”, “an”, and “the” mean “one or more.” For example, “an antigen” should be interpreted to mean “one or more antigens.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, hamsters, ferrets, rabbits, rats, mice, horses, cattle, cows, and the like. Avian species, such as chickens, geese, turkeys, ducks, etc., are also encompassed by the term.

As used herein, a “subject in need thereof” refers to a subject at risk for contracting an infection caused by a microorganism, or a subject infected with a microorganism, such as a viral, yeast, bacterial or parasitic infection. The term also encompasses a subject suspected of having or diagnosed as having an infection caused by a microorganism such as a viral, yeast, bacterial, or parasitic infection. As used herein, the phrase “in need thereof” indicates the state of the subject, wherein therapeutic or preventative measures are desirable. Such a state can include, but is not limited to, subjects having a disease or condition caused by an infection (e.g., viral, yeast, bacterial or parasitic infection), or at risk of any such infection. In some embodiments, a subject in need thereof includes a subject diagnosed with cancer, or at risk of cancer. As is known in the art, cancers express specific antigens which can be recognized and attacked by the immune system (e.g., tumor specific antigens, tumor specific neoepitopes [15], and tumor associated antigens). Categories of tumor antigens include products of mutated oncogenes and tumor suppressor genes, overexpressed or aberrantly expressed cellular proteins, tumor antigens produced by oncogenic viruses, altered cell surface glycolipids and glycoproteins, oncofetal antigens, cell type-specific differentiation antigens. A few non-limiting examples of tumor antigens that can be employed in the present methods and compositions include alphafetoprotien (AFP) found in germ cell tumors, and hepatocellular carcinoma; carcinoembyonic antigen (CEA) found in bowel cancers; CA-125 found in ovarian cancers; MUC-1 and/or epithelial tumor antigen (ETA) found in breast cancer; tyrosinase and/or melanoma-associated antigen (MAGE), found in malignant melanoma, and abnormal products of KRAS, TP53, found in various tumors, as well as individual-specific neoantigens.

As used herein, “viral load” refers to the amount of virus present in the blood, saliva, or other fluid or tissue sample of a patient or animal. Viral load is also referred to as viral titer or viremia. Viral load can be measured in variety of standard ways including by plaque assays or copy Equivalents of the viral nucleic acid, e.g., viral RNA (vRNA) genome per milliliter blood plasma (vRNA copy Eq/ml). This quantity may be determined by standard methods that include, for example, PCR or RT-PCR. In some embodiments, the composition disclosed herein (vaccines), after being administered to a subject in need thereof, result in a reduction in the viral load (upon subsequent challenge) in said subject compared to a control subject that did not previously receive a vaccine.

As used herein the term “vaccine” refers to a substance used to generate an immune response (e.g., induce an antibody response, activate T-cells, etc.), and that provides immunity against one or several diseases from the causative agent of the disease. A vaccine may comprise, for example, a weakened or killed form of the microbe, or a component isolated from a disease causing microbe (e.g., a surface protein or peptide or antigenic fragment thereof, a toxin molecule or a component of a toxin molecule produced or expressed by the microorganism), or a synthetic product that resembles it. In some embodiments, a vaccine comprises a protein or peptide derived from a microorganism of interest. In some embodiments, the vaccine is a viral vaccine (i.e., a vaccine used to ameliorate or prevent a disease caused by a natural viral infection). Exemplary viral vaccines include but are not limited to influenza vaccines, hepatitis A and B vaccines, human papilloma virus vaccine, zoster vaccine, smallpox vaccine, measles vaccine, rabies vaccine, poliovirus vaccine, Japanese encephalitis vaccine, rubella vaccine, rotavirus vaccine, yellow fever vaccine, varicella virus vaccine, lassa/machupo/junin/guanarito (and other hemorrhagic arenaviruses) vaccines, ebola virus vaccine, HIV vaccine, and coronavirus vaccine (e.g., SARS-CoV-2). In some embodiments the vaccine is a cancer vaccine (i.e., a vaccine to ameliorate or prevent cancer, which may or may not be caused by a virus, e.g., HPV). In some embodiments the compositions disclosed herein (e.g., VLPs that present antigenic protein) are formulated as a vaccine.

As used herein, the term “viral infection” refers to any undesired presence and/or replication of virus in a subject. Such undesired presence of virus may have a negative effect on the host subject's health and well-being. The term “viral infection” encompasses infections involving several species of viral pathogens as well as those involving a single viral species including mutant versions of viruses (e.g., naturally or non-naturally occurring variants). In some cases, the viral infection is caused by a virus selected from influenza virus, coronavirus (e.g., SARS-CoV-2), adenovirus, norovirus, rotavirus, and respiratory syncytial virus.

As used herein, the term “bacterial infection” refers to any undesired presence and/or growth of bacteria in a subject. Such undesired presence of bacteria may have a negative effect on the host subject's health and well-being. While the term “bacterial infection” should not be taken as encompassing the growth and/or presence of bacteria which are normally present in the subject, for example in the digestive tract of the subject, it may encompass the pathological overgrowth of such bacteria. The term “bacterial infection” encompasses infections involve several species of bacterial pathogens as well as those that involve a single bacterial species, including mutant versions of bacterial species (e.g., naturally or non-naturally occurring variants and metabolically inactive forms of bacteria resistant to antibiotics). Infections involving multiple species of bacterial pathogens are also known as complex, complicated, mixed, dual, secondary, synergistic, concurrent, polymicrobial, or co-infections.

As used herein, the term “yeast infection” or “fungal infection” are used interchangeably and refer to any undesired presence and/or growth of yeast in a subject. Such undesired presence of yeast may have a negative effect on the host subject's health and well-being. While the term “yeast infection” should not be taken as encompassing the growth and/or presence of yeasts which are normally present in the subject, for example as members of the normal flora of the skin, intestinal tract, oral and vaginal mucosa of the subject, it may encompass the pathological overgrowth of such yeasts. The term “yeast infection” encompasses infections involve several species of yeast as well as those that involve a single yeast species or mutant versions of yeasts (e.g., naturally or non-naturally occurring variants).

As used herein, the term “antigen” refers to an agent which is administered to a subject in need thereof in order to elicit an immune response against the antigen, which may include a protective immune response against the antigen such as in vaccination. Suitable antigens may comprise viruses, proteins (polypeptides, peptides), carbohydrates, lipids, nucleic acid, and any combination thereof. In some embodiments, an antigen is “multimeric,” comprising more than one identical epitope per VLP. As used herein, the term “epitope” means that part of the antigen to which an antibody binds or that part of the antigen that is recognized by B- and/or T-lymphocytes and/or antigen presenting cells (e.g. dendritic cells).

As used herein, the term “immune response” refers to a humoral immune response and/or cellular immune response leading to the activation or proliferation of B- and/or T-lymphocytes and/or antigen presenting cells. “Immunogenic” refers to an agent used to stimulate the immune system of a living organism, so that one or more functions of the immune system are increased and directed towards the immunogenic agent.

As used herein, the term “virus-like particle” (VLP) or “virus-like particle of a bacteriophage” refers to a virus-like particle (VLP) resembling the structure of a bacteriophage, being non-replicative and noninfectious, and lacking viral genes sufficient for infection, or at least the gene or genes encoding for the replication machinery of the bacteriophage, and typically also lacking the gene or genes encoding the protein or proteins responsible for viral attachment to or entry into the host. This definition also encompasses virus-like particles of bacteriophages, in which the aforementioned gene or genes are still present but inactive, and, therefore, also leading to non-replicative and noninfectious virus-like particles of a bacteriophage. VLPs include those of RNA bacteriophages and other viruses. While VLPs do not include the genes for infection and replication, in some embodiments, the genes encoding the viral coat proteins may be within a VLP.

In some embodiments, the VLPs described here include assemblies of the coat proteins of single-strand RNA bacteriophage (see e.g., RNA Bacteriophages, in The Bacteriophages. Calendar, R L, ed. Oxford University Press. 2005). The known viruses of this group attack bacteria as diverse as E. coli, Pseudomonas and Acinetobacter. Each possesses a highly similar genome organization, replication strategy, and virion structure. Thus, the present invention encompasses coat proteins of any of the following viruses, and is not limited to PP7, MS2, AP205, Qβ, R17, SP, PP7, GA, M11, MX1, f4, CbS, Cb12r, Cb23r, 7s and f2 RNA bacteriophages. A VLP is typically a capsid structure formed from the self-assembly of one or more subunits of a bacteriophage coat protein (CP). In some embodiments, the VLP capsid structure is formed from the self-assembly of coat protein single-chain dimers or coat protein monomers; in some embodiments, the coat protein is assembled from trimers, e.g., CP chains A, B, and C. (See e.g., [24], incorporated herein by reference in its entirety). The information required for assembly of the icosahedral capsid shell of this family of bacteriophage is contained entirely within coat protein itself. For example, purified coat protein can form capsids in vitro in a process stimulated by the presence of RNA. Moreover, coat protein expressed in cells from a plasmid assembles into a virus-like particle in vivo. A non-limiting example of coat protein includes the AP205 Acinobacter phage coat protein (NCBI Ref. No. NP_085472.1) shown below (SEQ ID NO: 28).

| | |
|---|---|
| 1 | mankpmqpit stankivwsd ptrlsttfsa sllrqrvkvg iaelnnvsgq yvsvykrpap |
| 61 | kpegcadacv impnenqsir tvisgsaenl atlkaeweth krnvdtlfas gnaglgfldp |
| 121 | taaivssdtt a |

It is understood that the present invention is not intended to be limited by a specific coat protein or its amino acid sequence; variants of such sequences, as well as synthetic coat proteins are also encompassed by the present disclosure.

As used herein, a VLP may be comprised of a self-assembled aggregation of one or more distinct antigen-CP fusion protein subunits, whereby each subunit is referred to as a “monomer” or together “monomers”. The exact number of antigen-CP fusion protein monomers that comprise a VLP may be dependent on factors such as but not limited to: the coat protein selected, the presence of additional recombinant genes or modification in the host cell, the identities of the antigen sequences linked to the CP to form the fusions, and whether the antigen sequences are linked to (i) the N-terminus of the CP, (ii) the C-terminus of the CP, or (iii) both the N-terminus and C-terminus of the CP. In the case of two or more distinct antigen-CP fusion protein monomers that comprise a VLP, the exact ratio of each distinct antigen-CP fusion protein in a VLP may vary dependent on factors such as but not limited to: the coat protein selected, the presence of additional recombinant genes or modification in the host cell, the identities of the antigen sequences linked to the CP to form the fusions, and whether the antigen sequences are linked to (i) the N-terminus of the CP, (ii) the C-terminus of the CP, or (iii) both the N-terminus and C-terminus of the CP. The number of antigen-CP fusion protein monomers in a VLP may be modulated.

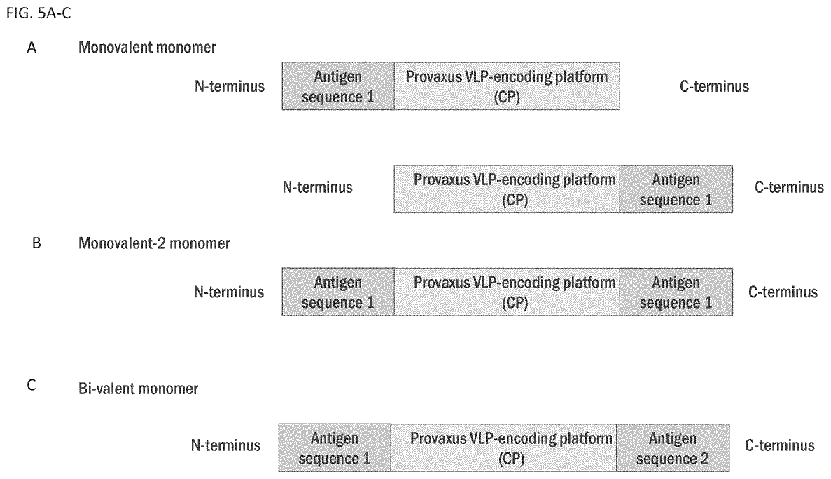

The term “valency”, as used herein, refers to the number of distinct antigens displayed by one or more antigen-CP fusion protein monomers expressed in a probiotic cell, regardless of whether those monomers are assembled into a VLP. The term “monovalent” refers to the case where only one distinct antigen is expressed. The term “multivalent” refers to the case where two or more distinct antigens are expressed. In one embodiment, an antigen-CP fusion protein monomer may have a single antigen sequence linked to one end (either the C-terminus or the N-terminus) of the CP. When only one such fusion protein is expressed then only one distinct antigen is displayed, and thus the valency is one and such a fusion is referred to herein as a “monovalent monomer” (FIG. 5A). In an alternative embodiment the antigen-CP fusion protein monomer may have two identical antigen sequences whereby each identical antigen sequence is linked to a different end (each of the C-terminus and N-terminus) of the CP. When only one such fusion protein is expressed then only one distinct antigen is displayed, and thus valency is one (albeit in two instances per monomer) and such a fusion is referred to herein as a “monovalent-2 monomer” (FIG. 5B). In yet another embodiment the antigen-CP fusion protein monomer may have two different antigen sequences whereby each antigen sequence is linked to a different end (each of the C-terminus and N-terminus) of the CP. When only one such fusion protein is expressed then two distinct antigens are displayed, and thus valency is two and such a fusion is referred to herein as a “bi-valent monomer” (FIG. 5C). Monovalent monomers, monovalent-2 monomers, and bi-valent monomers can be expressed individually or in any combination. A probiotic cell that expresses a single monovalent monomer will be monovalent with respect to the number of antigens displayed; and, likewise, if such monomers expressed by a probiotic cell assemble into VLPs, then the resulting VLPs will be monovalent. A probiotic cell that expresses a single monovalent-2 monomer will also be monovalent with respect to the number of antigens displayed; and, likewise, if such monomers expressed by a probiotic cell assemble into VLPs, then the resulting VLPs will be monovalent. A probiotic cell that expresses a single bi-valent monomer will be bi-valent with respect to the number of antigens displayed; and, likewise, if such monomers expressed by a probiotic cell assemble into VLPs, then the resulting VLPs will be bi-valent. A probiotic cell that expresses together any combination of monovalent, monovalent-2, and bi-valent monomers will display two or more distinct antigens, and the valency will be equal to the number of distinct antigens displayed; and, likewise, if such combinations of monomers expressed by a probiotic cell assemble into VLPs, then the resulting VLPs will be multivalent, with a valency equal to the number of distinct antigens displayed.

By way of example but not by way of limitation, the AP205 coat protein, which is exemplified herein, typically assembles a VLP comprising 180 coat protein monomers that self-assemble into 90 dimers and subsequently into an isohedral macromolecular complex. Thus, in an embodiment comprising a fusion of one antigen with the AP205 coat protein (wherein the single antigen is linked to one end of the coat protein), a valency of one can be expected in an assembled VLP. Alternatively, in an embodiment comprising a fusion of two antigens with the AP205 coat protein (wherein the one antigen is linked to one end of the coat protein and the other antigen is linked to the other end of the coat protein), due to the nature of AP205 assembly, a valency of two can be expected.

As another example, a single probiotic host may comprise two different recombinant constructs that express viral coat protein. The first construct may include the coat protein alone; the second construct may include the coat protein fused to a single antigen. Thus an assembled VLP could include some percentage of coat protein with the antigen, and some percentage of coat protein without the antigen. As an example, if both constructs are driven by the same promoter, the amount of protein produced by each would be expected to be equal, and the valency of an assembled VLP would be expected to be about 50%. If the promoter strength differed between the constructs, for example, if one promoter was inducible and one constitutive, then the number of monovalent monomers, monovalent-2 monomers, and/or bi-valent monomers that are contained within a VLP could be modulated.

Immunogenic compositions according to the present disclosure comprise VLPs which are, in some embodiments, are highly antigen-presenting. As used herein, highly antigen-presenting valency refers to a VLP (or collection of coat protein-antigen fusions) wherein at least about 50%, 60%, 70%, 805, 90% 95%, 98%, 99% or 100% of the coat proteins (either in the VLP or in a sample of coat proteins) display an antigen. The level of VLP antigen presentation may be determined by methods well known in the art. Non-limiting methods include crystal structure analysis (e.g., analyzing N- and/or C-termini, and/or surface elements, such as loops), computational modeling based on related crystal structures, cryogenic electron microscopy, and atomic force microscopy.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For purposes of this disclosure, “treating” or “treatment” describes the management and care of a patient for the purpose of combating the disease, condition, or disorder. The terms embrace both preventative, i.e., prophylactic, and palliative treatment. “Treating” includes the administration of a composition of the present invention to prevent the onset of the symptoms or complications, alleviating the symptoms or complications, or eliminating the disease, condition, or disorder. The term “treat” and words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of this disclosure can provide any amount of any level of treatment or prevention of disease in a subject. Furthermore, the treatment or prevention provided by the inventive method can include treatment or prevention of one or more conditions or symptoms of the disease or disease state, e.g., bacterial, viral, parasite or foreign antigen such as prion or venom, or infection, or cancer being treated or prevented. Also, for purposes herein, “prevention” can encompass delaying the onset of the disease, or a symptom or condition thereof.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease or to an amount that is effective to protect against the contracting or onset of a disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment outcome. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

Thus, in some embodiments, an “effective amount” may be used to describe a number of VLP's or an amount of a VLP-containing composition which, in context, is used to produce or effect an intended result, whether that result relates to the prophylaxis and/or therapy of an infection or infection-related disorder or disease state, including a viral or bacterial infection or as otherwise described herein. In some embodiments, an “effective amount” may refer to a number of engineered, therapeutic, probiotic microorganisms of the present disclosure (e.g., modified to produced antigenic VLPs), or an amount of a composition comprising such microorganisms that results in the prophylaxis and/or therapy of an infection or infection-related disorder or disease state, including a viral, yeast, bacterial, or parasitic infection, a cancer, or as otherwise described herein.

As used herein, the term “probiotic” refers to organisms, generally bacteria, which are considered to be beneficial rather than detrimental to their animal host. Methods and compositions disclosed herein may include engineering a probiotic microorganism, and administering the engineered organism to a subject in a variety of forms, for example, as a live culture, killed, as a spore, or in lyophilized form. It is now a popular concept that the accumulation of probiotic organisms in the gut is beneficial to the general health of the host organism and there are reports which indicate that the administration of probiotics is useful in the treatment of intestinal disease, as well as other diseases and conditions. In some embodiments of the present disclosure, probiotic bacteria are utilized as delivery vectors for oral vaccines. Exemplary probiotic bacteria include, without limitation Lactobacillus, Saccharomyces, Bifidobacterium, Streptococcus, Escherichia coli, Bacillus, Leuconostoc, Pediococcus, Lactococcus, Aerococcus, Carnobacterium, Enterococcus, Oenococcus, Sporolactobacillus, Teragenococcus, Vagococcus, Weisella and other such bacteria related by genome sequence.

In some embodiments, lactic acid bacterial are used. Lactic acid bacteria (LAB) comprise a group of Gram-positive bacteria that include, for example, species of Lactobacillus, Leuconostoc, Pediococcus, Lactococcus, Streptococcus, as well as the more peripheral Aerococcus, Carnobacterium, Enterococcus, Oenococcus, Sporolactobacillus, Teragenococcus, Vagococcus, and Weisella. In some embodiments, a Lactobacillus species is used. By way of example, but not by way of limitation, exemplary Lactobacillus species include Lactobacillus acidophilus, Lactobacillus crispatus, Lactobacillus gasseri, Lactobacillus delbreuckii, Lactobacillus rhamnosus, Lactobacillus salivarius, Lactobacillus paracasei, Lactobacillus reuteri, Lactobacillus bulgaricus, Lactobacillus casei, Lactobacillus lactis, Lactobacillus plantarum, Lactobacillus rhamnosus, Bifidobacterium longum, Bifidobacterium breve, Bifidobacterium lactis; and the heterofermentative species, Lactobacillus reuterior and Lactobacillus fermentum, and other such bacteria related by genome sequence. Additional Lactobacillus species include ATCC 53544, ATCC 53545, and ATCC 4356. For the sake of conciseness, the methods and compositions disclosed herein are exemplified using Lactobacillis acidophilus. However, it is to be understood that the invention is not so limited and that any probiotic microorganism that can be orally administered, and that is known to colonize the gut can be used engineered as described herein to produced VLPs and/or antigenic VLPs.

As used herein, the term “recT,” refers to the gene or its gene product. It is known in the art that the recT gene product binds to single-stranded DNA and also promotes the renaturation of complementary single-stranded DNA [17]. The recT protein functions in recombination and has a function similar to that of lambda redB [18]. In some embodiments, the recT gene (or redB gene, or other similarly functioning gene) may be included in a plasmid or vector to enhance or aid recombination of a transcription template into a host genome.

### Polynucleotides

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “promoter” refers to a cis-acting DNA sequence that directs RNA polymerase and other trans-acting transcription factors to initiate RNA transcription from the DNA template that includes the cis-acting DNA sequence. Promoters may include eukaryotic promoters which function in eukaryotic cells and prokaryotic promoters which function in prokaryotic cells.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered expression templates include nucleic acids composed of DNA or RNA. Suitable sources of nucleic acid for use in an expression template include genomic DNA, cDNA and RNA that can be converted into cDNA. The genomic DNA, cDNA and RNA can be from host cell or virus origins and from any species, including extant and extinct organisms. By way of example, in some embodiments, a transcription template is present in a vector, such as a plasmid vector and comprises a DNA vaccine-encoding platform sequence, including one or more of the following: a promoter, an antigen sequence, a linker or hinge sequence, a VLP sequence, a his-tag or other detectable marker, an immunostimulatory sequence, and a terminator. In some embodiments, the expression template is flanked by integration sequences.

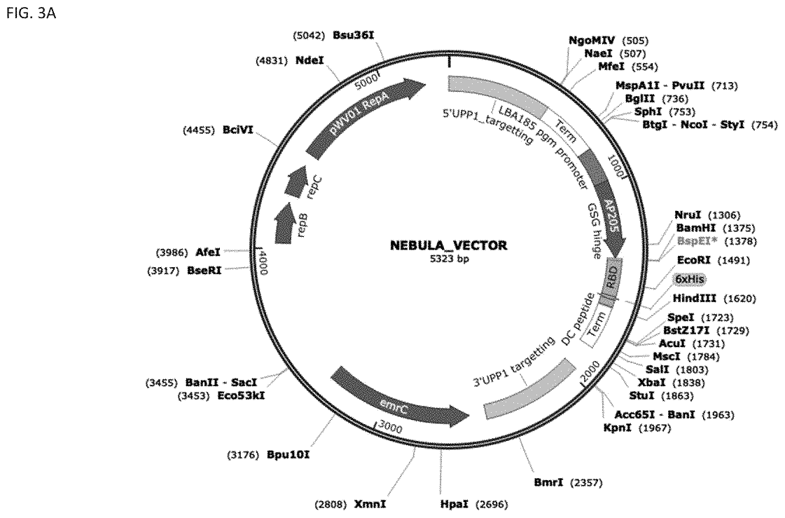

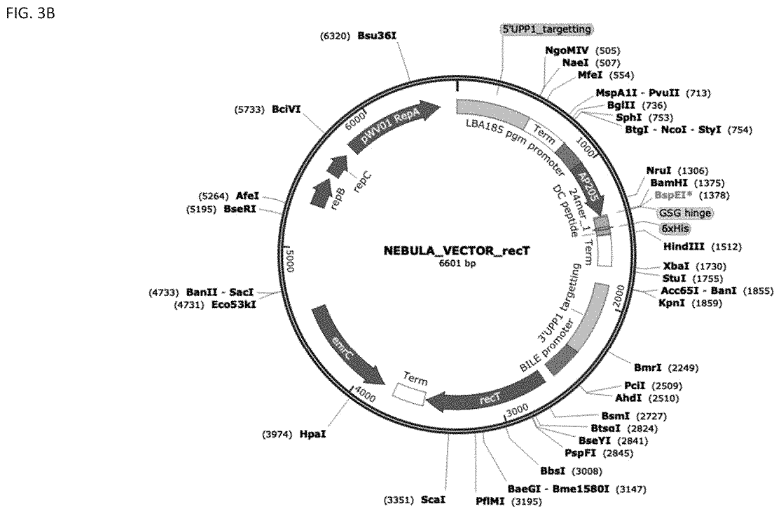

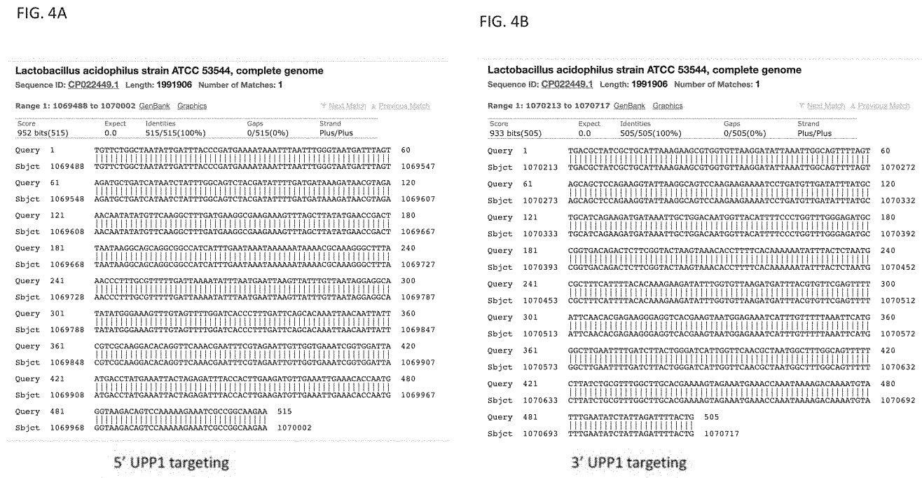

As used herein, the term “integration sequences” refer to sequences that facilitate site-directed insertion of heterologous nucleic sequence into a host genome. Exemplary integration sequences are preferably between a stop codon and a terminator, and downstream of genes with high constitutive or inducible expression. For example, integration sequences in Lactobacillus acidophilus include, but are not limited to upp sequences (e.g., as shown in FIG. 4), slpA sequences (e.g., LBA0169), eno sequences (e.g., LBA0889) and lacZ sequences (e.g., LBA1462). (See e.g., [30], incorporated herein by reference in its entirety). Orthologous genes in other bacteria, as well as genes with similarity in their arrangement of surrounding genes are also suitable loci for integration of expression templates of the present disclosure. (see e.g., FIG. 2; FIG. 3A-B).

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to a vehicle by which nucleic acid (e.g., DNA) can be introduced into a host organism or host tissue, including but not limited to integration into the genome of host cells. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the coat proteins and the coat protein-antigen fusion proteins disclosed herein). As used herein, “heterologous protein” refers to a protein that is not native or endogenous to the organism in which it is being expressed. The recombinant nucleic acid typically includes cis-acting elements for expression of the heterologous polypeptide, although in some cases the vector may introduce the heterologous polypeptide into the host genome such that endogenous host genomic cis-acting elements are used for the heterologous polypeptide expression. A vector may comprise an expression vector.

### Polypeptides, Peptides and Proteins

As used herein, the terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include conservative amino acid substitutions and/or non-conservative amino acid substitutions relative to a reference amino acid sequence. For example, a variant polypeptide may include conservative amino acid substitutions and/or non-conservative amino acid substitutions relative to the wild-type polypeptide. “Conservative amino acid substitutions” are those substitutions that are predicted to interfere least with the properties of the reference polypeptide. In other words, conservative amino acid substitutions substantially conserve the structure and the function of the reference protein. The following table provides a list of exemplary conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Alu |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

In contrast, “Non-conservative amino acid substitutions” are those substitutions that are predicted to interfere most with the properties of the reference polypeptide. For example, non-conservative amino acid substitutions may not conserve the structure and/or the function of the reference protein (e.g., substitution of a polar amino acid for a non-polar amino acid and/or substitution of a negatively charged amino acid for a positively charged amino acid).

A “deletion” refers to a change in the amino acid or nucleotide sequence that results in the absence of one or more amino acid residues or nucleotides relative to a reference sequence. A deletion may remove at least 1, 2, 3, 4, 5, 10, 20, 50, 100, or 200 amino acids residues or nucleotides. A deletion may include an internal deletion or a terminal deletion (e.g., an N-terminal truncation and/or a C-terminal truncation of a reference polypeptide).

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively.

In some embodiments, a fragment may comprise at least about 5, 10, 15, 20, 25, 30, 40, 50, 60, 70, 80, 90, 100, 150, 250, or 500 contiguous amino acid residues of a reference polypeptide. In some embodiments, a fragment may have a length within a range bounded by any value selected from 5, 10, 15, 20, 25, 30, 40, 50, 60, 70, 80, 90, 100, 150, 250, or 500, 1000, or 2000, etc., contiguous amino acid residues of a reference polypeptide. A fragment may comprise a percentage of a reference polypeptide. For example, a fragment may include contiguous amino acids that comprise about 5%, 10%, 15%, 20%, 25%, 30%, 40%, 50%, 60%, 70%, 80%, 90% 95%, 97%, 98%, or 99% of a reference polypeptide. Fragments may be preferentially selected from certain regions of a molecule. The term “at least a fragment” encompasses the full-length polypeptide.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” for example, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures, or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide.

As used herein the term “fusion protein” refers to a protein comprising at least two domains that are encoded by separate genes that have been joined so that they are transcribed and translated as a single unit, producing a single polypeptide. By way of example, the antigen-coat protein fusions of the present disclosure comprise an antigenic polypeptide fused to a bacteriophage coat protein (CP) (e.g., at the 5′ or 3′ end of a bacteriophage AP205 coat or “cap” protein). Such fusions may optionally include one or more linker sequences joining the antigenic peptide and the CP, one or more peptide tags, e.g., a His tag, and one or more immunostimulatory peptides.

### Probiotic Compositions

Disclosed herein is a novel vaccine platform comprising an engineered, probiotic microorganism modified to produce bacteriophage coat protein or a fusion protein comprising one or more antigens, e.g., multivalent antigens, linked to a bacteriophage coat protein. The coat proteins self-assemble, resulting in virus-like particles (VLPs) that present the antigenic protein on their outer surface.

When orally ingested by a subject, in some embodiments, the modified probiotic will colonize the gut and produce VLPs displaying an antigen. These VLPs will stimulate the subject's immune system, resulting in a protective immune response against later challenge by a microorganism harboring the same or similar antigen, or serve to attack an existing challenge, such as a tumor or a current infection. In some embodiments, the probiotic is modified to express only a coat protein (not fused to or linked to an antigen). Thus, in some embodiments, the probiotic will produce VLPs that modulate the immune system to the benefit of the subject. While the advantages of stimulating the immune system with an antigenic VLP are self-evident, the non-antigenic VLP will also generally stimulate the subject's immune system, providing several benefits to the subject.

Previous studies show that VLPs can activate the immune system by acting as an adjuvant to stimulate T helper type 1 (Th1) lymphocytes, and that such activation can be enhanced up to 1,000-fold when nonspecific RNAs from the bacterial host are encapsidated in comparison to empty VLPs [45]. Based on these results, it is expected that VLPs produced in probiotic bacteria will result in a general stimulation of the immune system, which in turn may result in bolstering resistance to infections from a variety of pathogens.

### Engineered Probiotic Microorganisms

The microorganisms of the present disclosure are modified (engineered) to produce a bacteriophage coat protein (CP) or a fusion protein comprising an antigenic protein linked to a bacteriophage coat protein. While the microorganisms may be modified to include an expression vector (e.g., a plasmid expression vector) to produce the fusion protein, preferably, the microorganism is modified to integrate a nucleic acid sequence encoding the CP or the CP-antigen fusion protein into the microorganism's genome. In some embodiments, microorganisms may be engineered with multiple transcription templates; the transcription templates may comprise the same or different proteins (e.g., CP proteins, and/or CP-antigen fusion proteins) for expression. The compositions disclosed herein are not limited by the method of genetic engineering employed, and any suitably means of stably integrating nucleic acid into the probiotic microorganism of choice is acceptable. Exemplary, non-limiting methods are outlined below.

Methods of integrating a nucleic acid sequence into a host genome are well known in the art; the addition of DNA conferring new or altered properties to microorganisms has underpinned biotechnology for decades.

As mentioned above, DNA can be added to microorganisms using replicative plasmids. Exemplary constructs for either inducible or constitutive expression from the plasmid can be constructed, e.g., as described in [25]. Typically, such systems of expression tend to be unstable, limiting their applied utility. Accordingly, methods have been developed to stably incorporate DNA molecules inside the cell, usually into a host chromosome.

With respect to random insertion, the phage Mu-driven transposition system, which can randomly insert the Mu DNA into the bacterial chromosome, has been widely applied to in vitro DNA transposition. Its function depends on the formation of transposome, a complex of Mu coding transposase, MuA, and DNA in the cell. Once formed, it can induce DNA cleavage and DNA strand transformation. (See e.g., [42]).

With respect to site specific integration (also termed “gene replacement”), the CRISPR-Cas systems can also be used for gene integration. The CRISPRs include of an array of a 30-40 bp short, direct repeat sequence that can be transcribed into the precursor crRNA (precrRNA) and the transactivating crRNA (tracrRNA). Another element of the system, the guide RNA (gRNA), is a fusion of tracrRNA and mature crRNA. The gRNA functions to bring together target and enzyme by guiding the RNA-guided DNA endonuclease Cas9 to cleave the gene target. CRISPR-Cas is highly versatile and can be applied to a variety of cells including various probiotics bacterial strains. (See e.g., [16]).

Site-specific integration can also be accomplished via homologous recombination systems using linear DNA for organisms such as yeast and naturally competent bacterial like Bacillus subtilis. Alternatively, an integrative plasmid bearing the homologous recombination construct can be used. As plasmids are circular, recombination events (e.g., a single cross-over or a double cross-over event) between the plasmid and the host genome can integrate the entire plasmid, or selected portions of the plasmid, into the chromosome.

By way of example, the λ, Red system is one of the most practical and widely utilized methods. It contains three essential proteins including Exo, Beta, and Gam from I-bacteriophage which can apply double-stranded DNA (dsDNA) or single-stranded DNA (ssDNA) into a specific chromosomal target. When combined with site-specific recombinase (SSR) systems including Cre/loxP and Flp/FRT, λ, Red system can manipulate almost any genetic alteration. (See e.g., [16], incorporated herein by reference in its entirety.)

Additional options for sight-directed gene replacement include systems that do not result in the host organism being labeled (e.g., antibiotic resistance) for selection (see e.g., [23]; [30] incorporated herein by reference in their entireties). An exemplary system includes the use of vector and the upp-counterselective gene replacement system (see e.g., [41] incorporated herein by reference in its entirety) and modifications thereof. By way of example only, FIGS. 2 and FIGS. 3A and 3B provide schematics of vectors useful to integrate a sequence of interest into a probiotic microorganism.

FIG. 2 provides a general schematic of a plasmid vector for homologous recombination, taking advantage of the upp-counterselective gene replacement system, and recT activity. The plasmid vector includes an expression template (e.g., a promoter, a sequence encoding the antigen-CP, and a terminator).

FIG. 3A and 3B provide non-limiting working examples of one plasmid embodiment for homologous recombination.

In some embodiments, a transcription template (e.g., the markerless (or unlabeled) DNA vaccine-encoding platform sequence) is encoded on a plasmid based on the pWV01 origin of replication that can shuttle between gram-positive and gram-negative bacteria and also contains an optional L. reuteri recT gene under the L. acidophilus LBA1432 promoter (bile-inducible, (see e.g., [28], incorporated herein by reference in its entirety) for increasing the efficiency of stable integration of the vaccine platform DNA sequence into the genome of the host, e.g., L. acidophilus.

As used herein “markerless” or “unlabeled” refer to an expression construct that is integrated into a host genome or is otherwise expressed or expressible in a host genome (e.g., via a plasmid or vector), that does not include a selectable marker (e.g. antibiotic resistance).

In some embodiments (e.g., as shown in FIG. 3), the genome-integrating markerless (or unlabeled) DNA vaccine-encoding platform (e.g., a transcription template) sequence includes the following (from the 5′ to the 3′ end): genome targeting sequence (>500 bases 5′ to the uracil phosphoribosyltransferase encoding gene (upp) open reading frame (ORF)), followed by the L. acidophilus-specific bi-directional terminator, the constitutive LA185, pgm promoter or other constitutive promoter for expression of the AP205-protein-coding sequence that contains a 3′ cloning site for a tri-peptide hinge sequence, then a 6xHis tag sequence, followed by the epitope-coding sequence and a dendritic cell-targeting peptide (DCpep), L. acidophilus bi-directional terminator, followed by the genome targeting sequence (>500 bases 3′ to the UPP open reading frame (ORF)) (see e.g., FIGS. 2 and 3; see [23, 25, 26, 27] incorporated herein by reference in their entireties). The genome insertion site, here, is the upp gene locus, which is universal in bacteria.

As described above, a markerless (or unlabeled) DNA vaccine-encoding platform sequence is inserted at the upp locus. However, the compositions and methods disclosed herein are not intended to be limited by integration site, and additional or alternative sites are encompassed. Thus, a markerless (or unlabeled) DNA vaccine-encoding platform sequence can be integrated at one or more additional or alternative genome loci. For example, intergenic regions in Lactobacillus acidophilus preferably between a stop codon and a terminator and downstream of genes with high constitutive or inducible expression can be replaced or interrupted with exogenous DNA, such as the markerless (or unlabeled) DNA vaccine-encoding platform sequence. As additional examples, intergenic locations downstream of slpA (LBA0169), ENO (LBA0889), lacZ (LBA1462), and slpX LBA0444-LBA0447 loci in Lactobacillus acidophilus can be replaced or interrupted with exogenous DNA (see .e.g., [29, 30], incorporated herein by reference in their entireties). Orthologous genes in several other bacteria, as well as some similarity in their arrangement of surrounding genes provide numerous options for integration of the markerless (or unlabeled) DNA vaccine-encoding platform sequence in different bacterial species.

### Transcription Template

The modified probiotic microorganisms disclosed herein comprise a transcription template, e.g., a markerless (or unlabeled) DNA vaccine-encoding platform sequence. The transcription template may also be present in a vector, such as plasmid vector, prior to incorporation into the microorganism. By way of example, in some embodiments, a transcription template includes at least one antigenic peptide sequence linked to a viral coat protein sequence to generate an antigen-CP fusion protein. In some embodiments, a transcription template includes a viral coat protein sequence that is not linked to an antigenic peptide. A transcription template may optionally include one or more of: a promoter sequence, a linker or hinge sequence, e.g., joining the antigenic peptide and the coat protein, a his-tag or other detectable protein marker, an immunostimulatory sequence, and at least one terminator. In some embodiments, the expression template is flanked by integration sequences.

A single probiotic microorganism may be modified to express and present a single antigenic peptide or multiple different antigenic peptides. By introducing different transcription templates (e.g., at different sites in the host microorganism's genome), with each transcription template encoding different antigenic peptide, different “species” of VLP's can be produced. In some embodiments, a VLP may be “mixed” and present multiple, different antigenic proteins.

The option to “tune” the expression level of one or more different CPs or antigen-CP fusions is also disclosed. By utilizing promoters having different strengths, and/or that are inducible versus constitutive within the microorganism, expression levels can be modulated to better fit the needs of the subject or to address the infectious situation. By way of example, multiple antigens being expressed at high levels (e.g., such that the modified probiotic microorganism produced a maximum amount of antigenic VLPs) may be warranted with respect to an infectious disease vaccine or a cancer vaccine - a situation in which a rapid, aggressive response is warranted. In other embodiments, a lower, constitutive level of expression may be warranted, for example, to sensitize a subject to an allergen [19]. Additionally, two copies of the gene encoding a VLP specific coat protein may be co-expressed at different levels (e.g. from a strong and a weak promoter) to achieve a mosaic composition of the native and antigen-coding CPs, which can promote and stabilize self-assembly of the VLP [20].

### Promoters and terminators

Any promoter expressed in the probiotic strain of choice may be used for the expression of the CP-antigen fusion, including any constitutive or inducible promoter that causes the expression of the CP or CP-antigen fusion. Typically, promoters are selected based on the level and timing of expression desired, and the organism which will be modified to express the protein. Those skilled in the art will be able to easily select and clone a suitable promoter into a vector for recombination into a compatible probiotic host microorganism. By way of example only, the L. acidophilus LA185, pgm promoter can be substituted for the L. acidophilus LBA1432 promoter, or another bile-inducible promoter may be selected for increasing the vaccine expression in the small intestine.

Likewise, any suitable termination sequence used by the probiotic microorganism of choice may be incorporated into the transcription template.

### Antigens

Any antigenic peptide may be used in the context of the present invention. Many bacterial and viral antigens are well known, and can be readily incorporated into the disclosed vaccine platform, and new antigenic peptides can be derived by methods well known in the art. Such methods include in vivo, in vitro, and in silico identification of antigenic moieties. For example, antigens may be identified by their reactivity to human sera. As is known in the art, antigenic peptides vary in length, and the methods of the present disclosure are suitably flexible so as not to be limited by antigen size or type. In some embodiments, an antigen-coat protein fusion may be expressed, but not assembled into a VLP. In some embodiments, VLP assembly is desired, and the antigen size (e.g., length of the amino acid sequence), and antigen position in the fusion relative to the CP may affect VLP assembly and antigen presentation in the assembled VLP. It is known in the art that different CPs have different configurations and VLP assembly characteristics. Accordingly, the design of a CP-antigen fusion construct requires consideration of known CP parameters to assist in the development and experimental testing of various constructs for VLP assembly and antigen presentation.

By way of example only and not by way of limitation, in some embodiments, wherein an AP205-antigen fusion is used and VLP assembly is desired, an antigenic peptide is about 6-2000, about 6-1000, about or 6-200 amino acids in length, or about 10, 20, 30, 40, 50, 60, 70, 80, 90, 100, 110, 120, 130, 140, 150, 160, 170, 180, 190 or about 200 amino acids in length. In some embodiments, an antigenic peptide is between about 1-180, about 20-170, about 30-160, about 40-150, about 50-140, about 60 - 130, about 70-120, about 80-110, or about 90-100 amino acids in length. In some embodiments, an antigenic peptide between is about 5-80, 5-70-5-60, 5-50, 5-40, 5-30, 5-20 or between about 5-10 amino acids in length. In some embodiments, an antigenic peptide is between about 10-80, 10-70-10-60, 10-50, 10-40, 10-30, 10-20 amino acids in length. In some embodiments, an antigenic peptide is between about 30-80, 30-70, 30-60, 30-50, or between about 30-40, amino acids in length. In some embodiments, an antigenic peptide is about 10-20, 10-30, 10-40, 10-50, 10-60, 10-70, 10-80, 10-90, 10-100 amino acids in length. In some embodiments, an antigenic peptide is about 200 to about 1000 amino acids in length, about 250-300, about 300-350, about 350-400, about 400-450, about 450-500, about 500-550, about 550-600, about 600-650, about 650-700, about 700-750, about 750-800 about 800-850, about 850-900, about 900-950, about 950-1000, about 250-950, about 300-900, about 350-850, about 400-800, about 450-750, about 500-700, about 550-650, or about 600-700, about 200-750, about 250-700, about 300-650, about 350-600, about 400-550, or about 450-500 amino acids in length. In some embodiments, the antigenic peptides are multivalent.

By way of example only, and not by way of limitation, antigenic peptides that could be used in the present methods and compositions include antigenic Spike Protein sequences from SARS-CoV-2 such as

(SEQ ID NO: 1)
YNYLYRLFRKSNLKPFERDISTEI,
(SEQ ID NO: 2)
LKPFERDISTEIYQAGSTPCNGVE,
(SEQ ID NO: 3)
TVCGPKKSTNLVKNKCVNFNFNGL,
(SEQ ID NO: 4)
YLYRLFRKSNLKPFERDISTEIYQAGSTPCNGVEGFNCYFPLQSYGFQPT
NGVGYQPYRV,
(SEQ ID NO: 5)
VIRGDEVRQIAPGQTGKIADYNYKLPDDFTGCVIAWNSNNLDSKVGGNYN
YLYRLFRKSN,
(SEQ ID NO: 6)
VRFPNITNLCPFGEVFNATRFASVYAWNRKRISNCVADYSVLYNSASFST
FKCYGVSPTKLNDLCFTNVYADSF,
(SEQ ID NO: 7)
RQIAPGQTGKIADYNYKLPD,
(SEQ ID NO: 8)
SYGFQPTNGVGYQ,
(SEQ ID NO: 9)
YAWNRKRISNCVA,
(SEQ ID NO: 10)
KPFERDISTEIYQ,
(SEQ ID NO: 11)
NYNYLYRLFR,
(SEQ ID NO: 12)
FNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFP
LQSYGFQPTNGVGYQPYRVVVLSFELLHA,
(SEQ ID NO: 13)
FNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFP
LQSYGFQPTYGVGYQPYRVVVLSFELLHA,
(SEQ ID NO: 14)
LKPFERDISTEIYQAGSTPCNGVK,
(SEQ ID NO: 15)
YLYRLFRKSNLKPFERDISTEIYQAGSTPCNGVKGFNCYFPLQSYGFQPT
NGVGYQPYRV,
and
(SEQ ID NO: 16)
FNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNL
DSKVGGNYNYLYRLFRKSNLKPFERDISTEIYQAGSTPCNGVKGFNCYFP
LQSYGFQPTNGVGYQPYRVVVLSFELLHA.

### Coat Proteins and VLPs

RNA-bacteriophage CPs have been shown to self-assemble into VLPs upon expression in a bacterial host. For exemplary purposes only, the AP205 phage CP is discussed. The AP205 phage VLP has demonstrated a high capacity and tolerance to foreign insertions and can tolerate long amino acid sequence additions at its CP N- and or C-termini without compromising capsid self-assembly (see e.g., [24]). As the N-terminus of one AP205 monomer CP in a dimer is in close proximity to the C-terminus of the other monomer and both termini are displayed on the surface of the VLP, both or at least one terminus can be used to display the peptide or polypeptide antigen sequence.

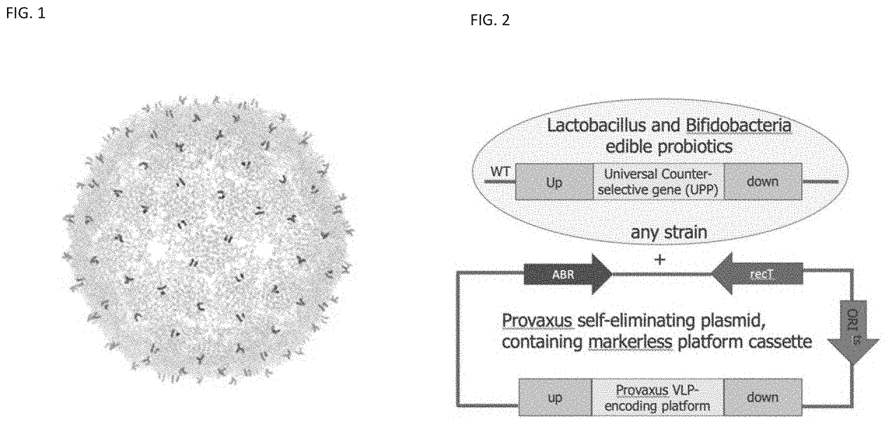

The structural integrity of the AP205 CP enables at least one antigen to be displayed on the VLPs in the form of an N-mer peptide (for example, from 6 to 200 or more amino acids in length) at either terminus of the AP205 CP (see e.g., FIG. 1). As previously discussed, the present disclosure is not limited to the AP205 CP; the present technology provides sufficient flexibility such that other viral CPs may be used with equal success and efficacy.

### Immunostimulatory Peptides

In some embodiments, an immunostimulatory peptide (e.g. see US2013/0287810A1, incorporated herein by reference in its entirety), also referred to as an immunostimulatory sequence, is fused to the VLP along with the selected antigen. For example, FYPSYHSTPQRP (SEQ ID NO: 17), which is a dendritic cell stimulating peptide [27], as well as toxoids such as diphtheria toxoid CRM197 or a derived peptide, tetanus neurotoxin TetX protein or a derived peptide such as VNNESSEVIVHK (SEQ ID NO: 18) may be used to enhance an immune response. In some embodiments, a host probiotic cell may be engineered with multiple expression templates, such that a first expression template expresses a CP-antigen fusion, and the second template expresses a CP-immunostimulatory peptide fusion. Promoters for the two different expression templates may be the same or different.

### Spacer Sequence

Optionally a spacer sequence may be included, e.g., between any of the functional domains of the transcription template. For example, a spacer may be included between the antigenic peptide sequence and the coat protein sequence, and/or between the immunostimulatory peptide sequence and the CP. Spacer sequences may be 2-20 amino acids in length, 5-15 amino acids in length, 8-11 amino acids in length, or smaller, e.g., 1-4 amino acids in length. In some embodiments, the spacer is useful to enhance antigen presentation on the outer surface of the self-assembled VLPs.

### Nutritional and Therapeutic Compositions

Disclosed herein are nutritional and therapeutic probiotic compositions comprising one or more probiotic microorganism engineered to produce a VLP, or a VLP presenting one or more antigens. In some embodiments, compositions comprising probiotic microorganism engineered to produce a VLP (non-antigen expressing) are useful as nutritional supplements. In some embodiments, compositions comprising probiotic microorganism engineered to produce VLPs expressing antigens are useful as therapeutics.

In some embodiments, the probiotic compositions are formulated for oral administration, for example, as a food product or a food supplement. By way of example but not by way of limitation, probiotic compositions may be formulated as a milk-based product, and may be provided in milk, yogurt, cheese, or ice cream. The food product may be formulated as a non-dairy product, such as a fruit-based product, or a soya-based product. Such foods products can be in solid or liquid/drinkable form. Further, the food product can contain all customary additives, including but not limited to, proteins, vitamins, minerals, trace elements, and other nutritional ingredients.

In some embodiments, a nutritional or therapeutic probiotic composition is formulated as a liquid, a powder, a capsule, a tablet, or a sachet for oral administration. In some embodiments, a capsule or tablet may include an enteric coating, and a probiotic composition may include one or more nutritionally or pharmaceutically acceptable carriers. In some embodiments, the carrier may be a capsule for oral administration. In such an embodiment, an outer housing of the capsule may optionally be made of gelatin or cellulose. Cellulose, starch, chitosan and/or alginate has the benefit of maintaining the formulation in intestinal fluid, disallowing premature breakdown in the upper gastrointestinal tract, so the product can reach the desired destination. Alternatively, the ingredients may be combined and formed into a tablet. In tablet form, cellulose starch, chitosan and/or alginate may also be present to act as a binder to hold the tablet together. Probiotic compositions may further comprise one or more excipients to facilitate the manufacturing process by preventing the ingredients from adhering to machines. Moreover, such excipients may render the capsule or tablet form easier to swallow and digest through the intestinal tract. The excipients may be a vegetable stearate, magnesium stearate, steric acid, ascorbyl palmitate, retinyl palmitate, or hyproxypropyl methylcellulose. Additional colors, flavors, and excipients known in the art may also be added. The formulated probiotic composition may be administered as formulated (e.g., as a capsule or tablet), or may be combined with food or drink for administration.

Nutritional or therapeutic probiotic compositions may include microorganisms provided in a variety of forms, including but not limited to lyophilized, in spore form (e.g., in suspension), as live cultures, as dead or inactivated microorganisms (e.g., heat inactivated or heat killed), or a combination thereof. By way of example, in some embodiments, compositions comprising live microorganisms may be orally ingested, and proceed to colonize the gut. The modified probiotic will then produce VLPs and stimulate the subject's immune system. In some embodiments, the microorganisms may be grown in culture and produce, or be induced to produce VLPs. The microorganisms comprising the VLPs may then be killed, lyophilized, or otherwise treated for further processing or storage. Any treatment methods or further processing should ideally leave at least a portion of the VLPs and/or antigenic proteins intact, regardless of the state or condition of the microorganism. In some embodiments, the VLPs are isolated.

In some embodiments, the microorganisms may be formulated and provided in nutritionally or therapeutically effective doses. In some embodiments, a nutritionally or therapeutically effective dose may comprise between about 1×10¹-1×10³ per dose, 1×10³-1×10²⁰, 1×10⁵-1×10¹⁵ microorganisms per dose; between about 1×10⁶-1×10¹⁴ microorganisms per dose; between about 1×10⁷-1×10¹³ microorganisms per dose; between about 1×10⁸-1×10¹² microorganisms per dose, between about 1×10⁹-1×10¹¹ microorganisms per dose; between about 1×10¹⁰-9×10¹⁰ microorganisms per dose; or about 3×10¹⁰ microorganisms per dose, or less than 10³ microorganisms per dose.

### Methods

Provided herein are method of treating or reducing incidence of a bacterial or viral infection in a subject in need thereof (i.e., vaccinating a subject). The methods include administering an effective amount of a therapeutic composition (e.g., an engineered probiotic and/or antigenic VLPs) of the present disclosure.

For therapeutic (e.g., vaccination) purposes, the exact dosage may be chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; mode of administration; and tolerance/response to therapy. For any active agent, the therapeutically effective dose can be estimated initially either in cell culture assays or in animal models, usually mice, rabbits, dogs, or pigs. The animal model is also used to determine a desirable concentration range and route of administration.

An effective does of a therapeutic probiotic composition may be administered to a subject in need thereof once per day, twice per day, three times per day, four times per day or more. In some embodiments an effective dose of a therapeutic probiotic composition is administered a single time, as a single dose. In some embodiments an effective dose of a therapeutic probiotic composition is administered daily, every other day, every third day, or once per week for at least about 1 week, at least about 2 weeks, about 3 weeks, 4 weeks, 5 weeks, 6 weeks, 7 weeks, 8 weeks, 9 weeks, 10 weeks 11 weeks, or at least about 12 weeks. In some embodiments, a therapeutic probiotic composition is administered periodically, as disease state, condition, or symptoms dictate. By way of example, but not by way of limitation, in some embodiments, an effective dose of a therapeutic probiotic composition is administered a single time, as a single dose.

In some embodiments, a therapeutic composition comprising an engineered probiotic such as L. acidophilus, is administered in combination with one or more additional active agents. By way of example, additional active agents include antacid such as salts of Calcium and or Magnesium, which neutralize stomach acidity. The additional active agent may be administered simultaneously with the probiotic composition (e.g., as part of the same formulation), or it may be administered separately, either at the same time or at a different time than the probiotic composition. Thus, in some embodiments, a subject in need thereof is administered a composition comprising a probiotic and one or more additional active agents.

The present methods are not intended to be limited by the mode of administration, and in some embodiments, suitable routes of administration may, for example, include oral, rectal, transmucosal, transnasal, intestinal, or parenteral delivery, including intramuscular, subcutaneous, and intramedullary injections as well as intrathecal, direct intraventricular, intravenous, intraperitoneal, intranasal, injections.

### EXAMPLES

### Example 1.

An antigenic Spike Protein sequence from SARS-CoV-2 is cloned into the plasmid vector presented in FIG. 3B. Exemplary antigenic Spike Protein sequences include the following:

| SEQ | | |
|---|---|---|
| ID No. | Protein | Exemplary Antigenic SARS-CoV-2 Spike Proteins/Peptides |
| 1 | S | YNYLYRLFRKSNLKPFERDISTEI |
| 2 | S | LKPFERDISTEIYQAGSTPCNGVE |
| 3 | S | TVCGPKKSTNLVKNKCVNFNFNGL |
| 4 | S | YLYRLFRKSNLKPFERDISTEIYQAGSTPCNGVEGFNCYFPLQSYGFQPTNGVGYQP |
| | | YRV |
| 5 | S | VIRGDEVRQIAPGQTGKIADYNYKLPDDFTGCVIAWNSNNLDSKVGGNYNYLYRLFR |
| | | KSN |
| 6 | S | VRFPNITNLCPFGEVFNATRFASVYAWNRKRISNCVADYSVLYNSASFSTFKCYGVS |
| | | PTKLNDLCFTNVYADSF |
| 12 | S | FNATRFASVYAWNRKRISNCVADYSVLYNSASFSTFKCYGVSPTKLNDLCFTNVYAD |
| | | SFVIRGDEVRQIAPGQTGKIADYNYKLPDDFTGCVIAWNSNNLDSKVGGNYNYLYRL |
| | | FRKSNLKPFERDISTEIYQAGSTPCNGVEGFNCYFPLQSYGFQPTNGVGYQPYRVVV |
| | | LSFELLHA |
| 13 | S-N501Y | FNATRFASVYAWNRKRISNCVADYSVLYNSASFSTFKCYGVSPTKLNDLCFTNVYAD |
| | | SFVIRGDEVRQIAPGQTGKIADYNYKLPDDFTGCVIAWNSNNLDSKVGGNYNYLYRL |
| | | FRKSNLKPFERDISTEIYQAGSTPCNGVEGFNCYFPLQSYGFQPTYGVGYQPYRVVV |
| | | LSFELLHA |
| 16 | S-E484K | FNATRFASVYAWNRKRISNCVADYSVLYNSASFSTFKCYGVSPTKLNDLCFTNVYAD |
| | | SFVIRGDEVRQIAPGQTGKIADYNYKLPDDFTGCVIAWNSNNLDSKVGGNYNYLYRL |
| | | FRKSNLKPFERDISTEIYQAGSTPCNGVKGFNCYFPLQSYGFQPTNGVGYQPYRVVV |
| | | LSFELLHA |
| 19 | S-S1/S2 | ASYQTQTNSPRRARSVASQS |

Additional Sequences for use in the disclosed methods and compositions

| SEQ | |
|---|---|
| ID NO. | Sequence |
| 29 | LKPFERDISTEIYQAGNTPCNGVE |
| 30 | IAWNSNNLDSKVGGNYNYRYRLFRKSNLKPFERDISTEIYQAG |
| | STPCNGVQGFNCYFPLQSYGFQP |
| 31 | VRQIAPGQTGNIADYNYKLPDDFTGCV |
| 32 | TEIYQAGSTPCNGVKGFNCYFPLQSYGFQPTYGVGYQPYRV |
| 33 | SKVGGNYNYRYRLFRKSNLKPFERDISTEIYQAGSTPCNGVKG |
| | FNCYGFPLQYGFQPTYGVGYQPYRV |
| 34 | GFNFSQILPDPSKPSKRSFIEDLLFNKVTLADAGFIKQYGDC |

The Spike Protein nucleic sequence is positioned adjacent to the AP205 coat protein (CP) nucleic acid sequence such that upon expression, a fusion protein is produced comprising the antigenic spike protein and coat protein. Each of the above ten spike protein-specific peptides is located at the C-terminus of the CP as individual clones. The vector based on rolling circle replication, and/or co-expressing recT is then used to introduce the antigen-VLP-encoding platform into Lactobacillus acidophilus by homologous recombination.

The vector is transformed into L. acidophilus using electroporation at 2.5 kV/cm, 25 uFD and 400 ohms and select on MRS plates containing 5 ug/m1 Erythromycin.

Induced transformants are plated on MRS plates containing only 5-fluorouracil (100 ug/ml) to select for genome integration at the UPP1 locus, at 37° C.,

Homologous integration is confirmed at upp1 by PCR, using primer sequences [TCGCAAGGACACAGGTTCAA (SEQ ID NO: 20) and GCATCTCCCAAACCAGGGAA (SEQ ID NO: 21); GTCCTGCACCTAAACCGGAA (SEQ ID NO: 22) and GCATCTCCCAAACCAGGGAA (SEQ ID NO: 23); TCGCAAGGACACAGGTTCAA (SEQ ID NO: 24) and TTCCGGTTTAGGTGCAGGAC (SEQ ID NO: 25)] and sequencing.

Recombinant bacteria are then tested for expression of antigenic VLPs. It is anticipated that the modified L. acidophilus will continue to grow, multiply, and produce antigenic VLPs. The expression level of antigen-presenting VLPs is determined using Western blotting with His-tag labeling and detection [21].

An evaluation of the VLPs is anticipated to show that VLP's display about 180 antigenic peptides when expressed as a monovalent monomer, or 360 antigenic peptides per VLP when expressed as a bi-valent monomer or monovalent-2 monomer. Additionally, electron microscopy analysis of the His-tag purified VLPs may be used to validate self-assembly in bacterial cells and to measure the particle sizes, which range from the estimated size of 30 nm to 60 nm diameter or larger, based on the molecular weight of the antigen peptide.

It is also anticipated that the antigenic proteins displayed by the VLPs are bound by antibody and antigen presenting cells and induce an immune response that includes cytotoxic T cells. To verify the latter, the modified probiotics will be tested in an animal model.

To demonstrate that the vaccine compositions of the present disclosure can stimulate an immune response, serum from vaccinated animals can be shown to bind His-tag purified VLPs with antigen-directed specificity and a vaccination experiment is conducted.

Six to seven weeks old transgenic ACE2 mice or hamsters susceptible to SARS-CoV-2 virus are provided oral doses of modified L. acidophilus. Control mice or hamsters received equal amounts of unmodified L. acidophilus. Six weeks later, blood samples are taken, and mice are then infected intranasally with live virus. In this experiment, a mouse-adapted SARS-CoV-2 model is used. A SARS-CoV-2 infection is simulated by a low inoculum of virus.

It is anticipated that the vaccinated mice and hamsters will exhibit VLPs in the GI track and blood samples and exhibit antibodies and T-cells directed against the SARS-CoV-2 spike protein antigen. It is also anticipated that the vaccinated mice and hamsters will show fewer symptoms, or no symptoms of viral infection as compared to the unvaccinated control mice and hamsters and have a lower or no detectable viral load as compared to control animals.

### REFERENCES

1. Rinninella, E.; Raoul, P.; Cintoni, M.; Franceschi, F.; Miggiano, G.A.D.; Gasbarrini, A.; Mele, M. C. What is the Healthy Gut Microbiota Composition? A Changing Ecosystem across Age, Environment, Diet, and Diseases. Microorganisms 2019, 7, 14. https://doi.org/10.3390/microorganisms7010014

2. Qin J, Li R, Raes J, et al. A human gut microbial gene catalogue established by metagenomic sequencing. Nature. 2010;464(7285):59-65. doi:10.1038/nature08821

3. Gerritsen, J., Smidt, H., Rijkers, G. T. et al. Intestinal microbiota in human health and disease: the impact of probiotics. Genes Nutr 6, 209-240 (2011). doi.org/10.1007/s12263-011-0229-7

4. Report of a Joint FAO/WHO Expert Consultation on Evaluation of Health and Nutritional Properties of Probiotics in Food Including Powder Milk with Live Lactic Acid Bacteria; Health and Nutritional Properties of Probiotics in Food including Powder Milk with Live Lactic Acid Bacteria, Food and Agriculture Organization of the United Nations, World Health Organization; Amerian Córdoba Park Hotel; Córdoba, Argentina; 1-4 Oct. 2001; www.fao.org/tempref/docrep/fao/meeting/009/y6398e.pdf

5. Markowiak P, Śliżewska K. Effects of Probiotics, Prebiotics, and Synbiotics on Human Health. Nutrients. 2017;9(9):1021. Published 2017 Sep. 15. doi:10.3390/nu9091021

6. Durack J, Lynch SV. The gut microbiome: Relationships with disease and opportunities for therapy. J Exp Med. 2019;216(1):20-40. doi:10.1084/jem.20180448

7. Verdugo-Meza A, Ye J, Dadlani H, Ghosh S, Gibson DL. Connecting the Dots Between Inflammatory Bowel Disease and Metabolic Syndrome: A Focus on Gut-Derived Metabolites. Nutrients. 2020;12(5):1434. Published 2020 May 15. doi:10.3390/nu12051434

8. Vitetta L, Saltzman ET, Thomsen M, Nikov T, Hall S. Adjuvant Probiotics and the Intestinal Microbiome: Enhancing Vaccines and Immunotherapy Outcomes [published correction appears in Vaccines (Basel). 2018 May 15;6(2):]. Vaccines (Basel). 2017;5(4):50. Published 2017 Dec. 11. doi:10.3390/vaccines5040050

9. Amicizia D, Micale R T, Pennati B M, et al. Burden of typhoid fever and cholera: similarities and differences. Prevention strategies for European travelers to endemic/epidemic areas. J Prev Med Hyg. 2019;60(4):E271-E285. Published 2019 Dec. 20. doi:10.15167/2421-4248/jpmh2019.60.4.1333

10. Smith I. Mycobacterium tuberculosis pathogenesis and molecular determinants of virulence. Clin Microbiol Rev. 2003;16(3):463-496. doi:10.1128/cmr.16.3.463-496.2003

11. Zimmermann P, Curtis N. Factors That Influence the Immune Response to Vaccination. Clin Microbiol Rev. 2019;32(2):e00084-18. Published 2019 Mar. 13. doi:10.1128/CMR.00084-18

12. Institute of Medicine (US) Vaccine Safety Committee; Stratton K R, Howe C J, Johnston R B Jr., editors. Adverse Events Associated with Childhood Vaccines: Evidence Bearing on Causality. Washington (DC): National Academies Press (US); 1994. 5, Diphtheria and Tetanus Toxoids. Available from: Causality. Washington (DC): National Academies Press (US); 1994. 5, Diphtheria and Tetanus Toxoids. Available from: www.ncbi.nlm.nih.gov/books/NBK236292/

13. WHO-UNICEF: Vaccine Handling BCG; extranet.who.int/ivb_policies/reports/vaccine handling.pdf

14. Vela Ramirez J E, Sharpe L A, Peppas N A. Current state and challenges in developing oral vaccines [published correction appears in Adv Drug Deliv Rev. 2018 Sep 3;:] [published correction appears in Adv Drug Deliv Rev. 2020;161-162:190-196]. Adv Drug Deliv Rev. 2017;114:116-131. doi:10.1016/j.addr.2017.04.008

15. The problem with neoantigen prediction. Nat Biotechnol 35, 97 (2017). doi.org/10.1038/nbt.3800

16. Jiang, et al., Targeting ideal oral vaccine vectors based on probiotics: a systematical view; Applied Microbiology and Biotechnology (2019) 103:3941-3953.

17. Hall SD, Kane MF, Kolodner RD. Identification and characterization of the Escherichia coli RecT protein, a protein encoded by the recE region that promotes renaturation of homologous single-stranded DNA. J Bacteriol. 1993 Jan;175(1):277-87. doi: 10.1128/jb.175.1.277-287.1993. Erratum in: J Bacteriol 1993 February;175(4):1211. PMID: 8416902; PMCID: PMC196123

18. Mosberg J A, Lajoie M J, Church G M. Lambda red recombineering in Escherichia coli occurs through a fully single-stranded intermediate. Genetics. 2010 Nov;186(3):791-9. doi: 10.1534/genetics.110.120782. Epub 2010 Sep. 2. PMID: 20813883; PMCID: PMC2975298

19. Yu W, Freeland DMH, Nadeau K C. Food allergy: immune mechanisms, diagnosis and immunotherapy. Nat Rev Immunol. 2016;16(12):751-765. doi:10.1038/nri.2016.111

20. Cielens I, Jackevica L, Strods A, Kazaks A, Ose V, Bogans J, Pumpens P, Renhofa R. Mosaic RNA phage VLPs carrying domain III of the West Nile virus E protein. Mol Biotechnol. 2014 May;56(5):459-69. doi: 10.1007/s12033-014-9743-3. PMID: 24570176

21. Nanoprobes.com products page: GoldiBlot™ www.nanoprobes.com/products/GoldiBlot.html

22. An Electron Microscopic Study of the Adherence of Lactobacillus Acidophilus to Human Intestinal Cells in Vitro, Food Microstructures, Vol 8 (1989), pp. 97-97, Scanning Microscopy International, Chicago Ill.; digitalcommons.usu.edu/foodmicrostructure/vol8/iss1/12

23. A general system for generating unlabelled gene replacements in bacterial chromosomes, Leenhouts K, Buist G, Bolhuis A, ten Berge A, Kiel J, Mierau I, Dabrowska M, Venema G, Kok J. A general system for generating unlabelled gene replacements in bacterial chromosomes. Mol Gen Genet. 1996 November 27;253(1-2):217-24. doi: 10.1007/s004380050315. PMID: 9003306. pubmed.ncbi.nlm.nih.gov/9003306

24. The True Story and Advantages of RNA Phage Capsids as Nanotools, Pumpens P, Renhofa R, Dishlers A, Kozlovska T, Ose V, Pushko P, Tars K, Grens E, Bachmann MF. The True Story and Advantages of RNA Phage Capsids as Nanotools. Intervirology. 2016;59(2):74-110. doi: 10.1159/000449503. Epub 2016 Nov. 10. PMID: 27829245. pubmed.ncbi.nlm.nih.gov/27829245-

25. Construction of vectors for inducible and constitutive gene expression in Lactobacillus, Duong T, Miller M J, Barrangou R, Azcarate-Peril M A, Klaenhammer T R. Construction of vectors for inducible and constitutive gene expression in Lactobacillus. Microb Biotechnol. 2011 May;4(3):357-67. doi: 10.1111/j.1751-7915.2010.00200.x. Epub 2010 Sep. 1. PMID: 21375708; PMCID: PMC3818994. pubmed.ncbi.nlm.nih.gov/21375708-

26. https://www.ncbi.nlm.nih.gov/gene/956335—AP205_3 coat protein [ Acinetobacter phage AP205 ]

27. Peptides Identified through Phage Display Direct Immunogenic Antigen to Dendritic Cells ,Tyler J. Curiel, Cindy Morris, Michael Brumlik, Samuel J. Landry, Kristiaan Finstad, Anne Nelson, Virendra Joshi, Christopher Hawkins, Xavier Alarez, Andrew Lackner, Mansour Mohamadzadeh, The Journal of Immunology Jun. 15, 2004, 172 (12) 7425-7431; DOI: 10.4049/jimmunol. 172.12.7425. www.jimmunol.org/content/172/12/7425

28. Characterization of a novel bile-inducible operon encoding a two-component regulatory system in Lactobacillus acidophilus, Pfeiler E A, Azcarate-Peril M A, Klaenhammer T R. Characterization of a novel bile-inducible operon encoding a two-component regulatory system in Lactobacillus acidophilus. J Bacteriol. 2007 July;189(13):4624-34. doi: 10.1128/JB.00337-07. Epub 2007 Apr. 20. PMID: 17449631; PMCID: PMC1913432. https://pubmed.ncbi.nlm.nih.gov/17449631-

29. Regulation of induced colonic inflammation by Lactobacillus acidophilus deficient in lipoteichoic acid, Mohamadzadeh M, Pfeiler E A, Brown J B, Zadeh M, Gramarossa M, Managlia E, Bere P, Sarraj B, Khan M W, Pakanati K C, Ansari M J, O'Flaherty S, Barrett T, Klaenhammer TR. Regulation of induced colonic inflammation by Lactobacillus acidophilus deficient in lipoteichoic acid. Proc Natl Acad Sci U S A. 2011 Mar 15;108 Suppl 1 (Suppl 1):4623-30. doi: 10.1073/pnas.1005066107. Epub 2011 Jan. 31. PMID: 21282652; PMCID: PMC3063598. pubmed.ncbi.nlm.nih.gov/21282652-

30. Directed Chromosomal Integration and Expression of the Rpeorter gene gusA3 in Lactobacillus, APPLIED AND ENVIRONMENTAL MICROBIOLOGY, October 2011, p. 7365-7371 Vol. 77, No. 20 0099-2240/11/$12.00 doi:10.1128/AEM.06028-11 Copyright © 2011, American Society for Microbiology. www.ncbi.nlm.nih.gov/pmc/articles/PMC3194874-

31. Bacteriophage translocation, Górski A, Wazna E, Dabrowska B W, Dabrowska K, Switala-Jeleń K, Miedzybrodzki R. Bacteriophage translocation. FEMS Immunol Med Microbiol. 2006 April;46(3):313-9. doi: 10.1111/j.1574-695X.2006.00044.x. PMID: 16553803. pubmed.ncbi.nlm.nih.gov/16553803 -

32. A bacteriophages journey through the human body, Barr J J. A bacteriophages journey through the human body. Immunol Rev. 2017 September;279(1):106-122. doi: 10.1111/imr.12565. PMID: 28856733. pubmed.ncbi.nlm.nih.gov/28856733-

33. Bacteriophages: Uncharacterized and Dynamic Regulators of the Immune System, Sinha A, Maurice C F. Bacteriophages: Uncharacterized and Dynamic Regulators of the Immune System. Mediators Inflamm. 2019 Sep. 8;2019:3730519. doi: 10.1155/2019/3730519. PMID: 31582898; PMCID: PMC6754933. pubmed.ncbi.nlm.nih.gov/31582898-

34. Lactobacillus mucosal vaccine vectors. 2018 May-Jun; 3(3): e00061-18. Published online 2018 May 16. www.ncbi.nlm.nih.gov/pmc/articles/PMC5956152-

35. Mucosal Delivery of Therapeutic and prophylactic molecules, Nature Reviews Microbiology, Nature Publishing Group, Vol. 6, May 2008, p349-362. www.nature.com/articles/nrmicro1840-

36. Dendritic Cell Targeting of Bacillus (PNAS), PNAS, Mar. 17, 2009, Vol. 106, No. 11, p. 4331-4336. www.pnas.org/content/106/11/4331-

37. U.S. Pat. No. 7,348,420 Klaenhammer, et al., Mar. 25, 2008.

38. U.S. Pat No. 10,815,291 Bolen, et al., Oct. 27, 2020.

39. U.S. Pat. No. 10,301,594 Kahvejian, et al., May 28, 2019.

40. U.S. Pat No 8,372,409 Mohamadzadeh, Feb. 12, 2013.

41. Goh, et al., Development and Application of a upp-Based Counterselective Gene Replacement System for the Study of the S-Layer Protein SlpX of Lactobacillus acidophilius NCFM; Applied and Environmental Microbiology, May 2009, 3093-3105.

42. Akhverdyan et al., Application of the bacteriophage Mu-driven system for the integration/amplification of target genes in chromosomes of engineered Gram-negative bacteria- mini review; Appl. Microbiol Biotechnol. 2011 91(4):857-871.

43. Zheng, D., Liwinski, T. & Elinav, E. Interaction between microbiota and immunity in health and disease. Cell Res 30, 492-506 (2020). https://doi.org/10.1038/s41422-020-0332-7.

44. Centers for Disease Control and Prevention; Vaccines and Preventable Diseases; Diphtheria, Tetanus, and Whooping Cough Vaccination: What Everyone Should Knowwww.cdc.gov/vaccines/vpd/dtap-tdap-td/public/index.html

45. Riedl P, Stober D, Oehninger C, Melber K, Reimann J, Schirmbeck R. Priming Thl immunity to viral core particles is facilitated by trace amounts of RNA bound to its arginine-rich domain. J Immunol. 2002 May 15;168(10):4951-9. doi: 10.4049/jimmuno1.168.10.4951. PMID: 11994446

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations that is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A modified probiotic microorganism comprising:
a nucleic acid sequence encoding a heterologous protein, the heterologous protein comprising:
(a) a viral coat protein; or
(b) a fusion of an antigenic peptide and a viral coat protein.

2. The modified probiotic microorganism of claim 1, wherein the probiotic microorganism comprises a bacteria selected from the group consisting of Lactobacillus, Saccharomyces, Bifidobacterium, Streptococcus, Escherichia coli, and Bacillus, Leuconostoc, Pediococcus, Lactococcus, Aerococcus, Carnobacterium, Enterococcus, Oenococcus, Sporolactobacillus, Teragenococcus, Vagococcus, Weisella and other such bacteria related by genome sequence.

3. The modified probiotic microorganism of claim 2, wherein the probiotic microorganism comprises a Lactobacillus selected from the group consisting of Lactobacillus acidophilus, Lactobacillus crispatus, Lactobacillus gasseri, Lactobacillus delbreuckii, Lactobacillus rhamnosus, Lactobacillus salivarius, Lactobacillus paracasei, Lactobacillus reuteri, Lactobacillus bulgaricus, Lactobacillus casei, Lactobacillus lactis, Lactobacillus plantarum, Lactobacillus rhamnosus, Bifidobacterium longum, Bifidobacterium breve, Bifidobacterium lactis, Lactobacillus reuterior and Lactobacillus fermentum and other such bacteria related by genome sequence.

4. The modified probiotic microorganism of claim 3 comprising Lactobacillus acidophilus.

5. The modified probiotic microorganism of claim 1, wherein the nucleic acid sequence encoding the heterologous protein is integrated into the genome of the microorganism or is encoded on a plasmid or a vector within the microorganism.

6. The modified probiotic microorganism of claim 1, wherein the nucleic acid sequence encoding the heterologous protein is integrated into the uracil phosphoribosyltransferase (upp) gene of the microorganism or at other suitable genome loci.

7. The modified probiotic microorganism of claim 1, wherein the microorganism expresses the heterologous protein, and wherein the expressed protein self-assembles to form virus-like particles (VLPs).

8. The modified microorganism of claim 7, comprising VLPs.

9. The modified probiotic microorganism of claim 8, wherein the heterologous nucleic acid encodes a fusion of an antigenic peptide and a viral coat protein, and wherein VLP valency is one or greater.

10. The modified probiotic microorganism of claim 1, wherein the heterologous nucleic acid encodes a fusion of an antigenic peptide and a viral coat protein, and wherein the expressed protein does not self-assemble to form VLPs.

11. The modified probiotic microorganism of claim 1, wherein the nucleic acid sequence encodes a fusion protein, the fusion protein further comprising one or more of the following:
(c) a linker sequence joining the antigenic peptide and coat protein;
(d) an immunostimulatory sequence.

12. The modified probiotic microorganism of claim 1, wherein the viral coat protein comprises one or more of the PP7, MS2, AP205, Qβ, R17, SP, PP7, GA, M11, MX1, f4, CbS, Cb 12r, Cb23r, 7s and f2 coat proteins.

13. The modified probiotic microorganism of claim 1, wherein the viral coat protein comprises the bacteriophage AP205 coat protein.

14. The modified probiotic microorganism of claim 1, wherein the microorganism is live in culture, in spore form, or inactivated.

15. The modified probiotic microorganism of claim 1, wherein the microorganism is dead or is lyophilized.

16. A nutritional or therapeutic composition comprising the modified probiotic microorganism of claim 1.

17. The composition of claim 16, formulated as a food or beverage or otherwise incorporated into the food supply.

18. The composition of claim 16, wherein the food or beverage comprises a dairy product.

19. The composition of claim 16, comprising milk, yogurt, cheese, ice cream.

20. The composition of claim 16, formulated as a capsule, powder, table, liquid, or sachet for oral administration.

21. The composition of claim 16, formulated for nasal, rectal, parenteral, or transmucosal delivery.

22. A method of vaccinating a subject comprising: administering an effective amount of the composition of claim 16 to the subject.

23. A method of providing nutritional supplementation to a subject, comprising: administering the composition of claim 16 to the subject.

24. The method of claim 22, wherein administration comprises oral administration.

25. The method of claim 22, wherein administration comprises nasal, rectal, parenteral, or transmucosal delivery.

26. The method 22, wherein an effective amount is provided as one or more doses.

27. The method of claim 26, wherein an effective amount is provided by administering multiple doses over the course of a week, two weeks, three weeks, or a month.

28. The method of claim 26, wherein an effective amount is provided as a single dose in a single administration.


SEQUENCE LISTING (34 sequences)

SEQ ID NO: 1 - protein, 24 aa, Artificial Sequence
  Synthetic- SARS-CoV-2 spike protein fragment
YNYLYRLFRKSNLKPFERDISTEI

SEQ ID NO: 2 - protein, 24 aa, Artificial Sequence
  Synthetic- SARS-CoV-2 spike protein fragment
LKPFERDISTEIYQAGSTPCNGVE

SEQ ID NO: 3 - protein, 24 aa, Artificial Sequence
  Synthetic- SARS-CoV-2 spike protein fragment
TVCGPKKSTNLVKNKCVNFNFNGL

SEQ ID NO: 4 - protein, 60 aa, Artificial Sequence
  Synthetic- SARS-CoV-2 spike protein fragment
YLYRLFRKSNLKPFERDISTEIYQAGSTPCNGVEGFNCYFPLQSYGFQPTNGVGYQPYRV

SEQ ID NO: 5 - protein, 60 aa, Artificial Sequence
  Synthetic- SARS-CoV-2 spike fragment
VIRGDEVRQIAPGQTGKIADYNYKLPDDFTGCVIAWNSNNLDSKVGGNYNYLYRLFRKSN

SEQ ID NO: 6 - protein, 74 aa, Artificial Sequence
  Synthetic- SARS-CoV-2 spike fragment
VRFPNITNLCPFGEVFNATRFASVYAWNRKRISNCVADYSVLYNSASFSTFKCYGVSPTKLNDLCFTNVYADSF

SEQ ID NO: 7 - protein, 20 aa, Artificial Sequence
  Synthetic- SARS-CoV-2 spike fragment
RQIAPGQTGKIADYNYKLPD

SEQ ID NO: 8 - protein, 13 aa, Artificial Sequence
  Synthetic- SARS-CoV-2 spike fragment
SYGFQPTNGVGYQ

SEQ ID NO: 9 - protein, 13 aa, Artificial Sequence
  Synthetic- SARS-CoV-2 spike fragment
YAWNRKRISNCVA

SEQ ID NO: 10 - protein, 13 aa, Artificial Sequence
  Synthetic- SARS-CoV-2 spike fragment
KPFERDISTEIYQ

SEQ ID NO: 11 - protein, 10 aa, Artificial Sequence
  Synthetic- SARS-CoV-2 spike fragment
NYNYLYRLFR

SEQ ID NO: 12 - protein, 179 aa, Artificial Sequence
  Synthetic- SARS-CoV-2 spike fragment
FNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHA

SEQ ID NO: 13 - protein, 179 aa, Artificial Sequence
  Synthetic- SARS-CoV-2 spike fragment
FNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHA

SEQ ID NO: 14 - protein, 24 aa, Artificial Sequence
  Synthetic- SARS-CoV-2 spike fragment
LKPFERDISTEIYQAGSTPCNGVK

SEQ ID NO: 15 - protein, 60 aa, Artificial Sequence
  Synthetic- SARS-CoV-2 spike fragment
YLYRLFRKSNLKPFERDISTEIYQAGSTPCNGVKGFNCYFPLQSYGFQPTNGVGYQPYRV

SEQ ID NO: 16 - protein, 179 aa, Artificial Sequence
  Synthetic- SARS-CoV-2 spike fragment
FNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHA

SEQ ID NO: 17 - protein, 12 aa, Artificial Sequence
  Synthetic- Dendritic cell stimulating peptide
FYPSYHSTPQRP

SEQ ID NO: 18 - protein, 12 aa, Artificial Sequence
  Synthetic- tetanus neurotoxin TetX derived peptide
VNNESSEVIVHK

SEQ ID NO: 19 - protein, 20 aa, Artificial Sequence
  Synthetic SARS-CoV-2 Spike fragment
ASYQTQTNSPRRARSVASQS

SEQ ID NO: 20 - DNA, 20 bp, Artificial Sequence
  Synthetic- upp PCR primer
tcgcaaggacacaggttcaa

SEQ ID NO: 21 - DNA, 20 bp, Artificial Sequence
  Synthetic- upp PCR primer
gcatctcccaaaccagggaa

SEQ ID NO: 22 - DNA, 20 bp, Artificial Sequence
  Synthetic- upp PCR primer
gtcctgcacctaaaccggaa

SEQ ID NO: 23 - DNA, 20 bp, Artificial Sequence
  Synthetic- upp PCR primer
gcatctcccaaaccagggaa

SEQ ID NO: 24 - DNA, 20 bp, Artificial Sequence
  Synthetic- upp PCR primer
tcgcaaggacacaggttcaa

SEQ ID NO: 25 - DNA, 20 bp, Artificial Sequence
  Synthetic- upp PCR primer
ttccggtttaggtgcaggac

SEQ ID NO: 26 - DNA, 515 bp, Lactobacillus acidophilus
tgttctggctaatattgatttacccgatgaaaataaatttaatttgggtaatgatttagtagatgctgatcataatctatttggcagtctacgatattttgatgataaagataacgtagaaacaatatatgttcaaggctttgatgaaggcgaagaaagtttagcttatatgaaccgacttaataaggcagcaggcggccatcatttgaataaataaaaaataaaacgcaaagggctttaaaccctttgcgtttttgattaaaatatttaatgaattaagttatttgttaataggaggcatatatgggaaagtttgtagttttggatcaccctttgattcagcacaaattaacaattattcgtcgcaaggacacaggttcaaacgaatttcgtagaattgttggtgaaatcggtggattaatgacctatgaaattactagagatttaccacttgaagatgttgaaattgaaacaccaatgggtaagacagtccaaaaagaaatcgccggcaagaa

SEQ ID NO: 27 - DNA, 505 bp, Lactobacillus acidophilus
tgacgctatcgctgcattaaagaagcgtggtgttaaggatattaaattggcagttttagtagcagctccagaaggtattaaggcagtccaagaagaaaatcctgatgttgatatttatgctgcatcagaagatgataaattgctggacaatggttacattttccctggtttgggagatgccggtgacagactcttcggtactaagtaaacaccttttcacaaaaaatatttactctaatgcgctttcattttacacaaagaagatatttggtgttaagatgatttacgtgttcgagttttattcaacacgagaagggaggtcacgaagtaatggagaaatcatttgtttttaaattcatgggcttgaattttgatcttactgggatcattggttcaacgctaatggctttggcagtttttcttatctgcgtttggcttgcacgaaaagtagaaatgaaaccaaataaaagacaaaatgtatttgaatatctattagattttactg

SEQ ID NO: 28 - protein, 131 aa, Artificial Sequence
  Synthetic- AP205 Acinobacter phage coat protein
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 29 - protein, 24 aa, Artificial Sequence
  Synthetic peptide
LKPFERDISTEIYQAGNTPCNGVE

SEQ ID NO: 30 - protein, 66 aa, Artificial Sequence
  Synthetic peptide
IAWNSNNLDSKVGGNYNYRYRLFRKSNLKPFERDISTEIYQAGSTPCNGVQGFNCYFPLQSYGFQP

SEQ ID NO: 31 - protein, 27 aa, Artificial Sequence
  Synthetic peptide
VRQIAPGQTGNIADYNYKLPDDFTGCV

SEQ ID NO: 32 - protein, 41 aa, Artificial Sequence
  Synthetic peptide
TEIYQAGSTPCNGVKGFNCYFPLQSYGFQPTYGVGYQPYRV

SEQ ID NO: 33 - protein, 68 aa, Artificial Sequence
  Synthetic peptide
SKVGGNYNYRYRLFRKSNLKPFERDISTEIYQAGSTPCNGVKGFNCYFPLQSYGFQPTYGVGYQPYRV

SEQ ID NO: 34 - protein, 42 aa, Artificial Sequence
  Synthetic peptide
GFNFSQILPDPSKPSKRSFIEDLLFNKVTLADAGFIKQYGDC